(19) United States
(12) Patent Application Publication
(10) Pub. No.: US 2023/0360801 A1
(43) Pub. Date: Nov. 9, 2023

(54) METHODS AND SYSTEMS OF IDENTIFYING INDIVIDUALS FOR PERIOPERATIVE NEUROCOGNITIVE DISORDER AND/OR POST-VIRAL COGNITIVE IMPAIRMENT

(71) Applicant: Cambridge Cognition Limited, Bottisham (GB)

(72) Inventors: Kiri Granger, Macclesfield (GB); Jennifer Barnett, Macclesfield (GB)

(73) Assignee: Cambridge Cognition Limited, Bottisham (GB)

(21) Appl. No.: 18/353,249

(22) Filed: Jul. 17, 2023

Related U.S. Application Data
(63) Continuation of application No. PCT/EP2022/050916, filed on Jan. 17, 2022.
(60) Provisional application No. 63/137,802, filed on Jan. 15, 2021.

(51) Int. Cl. A61K 31/164; G16H 50/30

(52) U.S. Cl. CPC G16H 50/30; A61K 31/164

(57) ABSTRACT

The present specification discloses systems, methods, and uses of identifying an individual at risk of a perioperative neurocognitive disorder or identifying or confirming a diagnosis of a post-viral cognitive impairment. The disclosed systems, methods and uses employ an episodic memory score, a working memory score, or both for an individual, optionally in conjunction with one or more additional risk-related factors from the individual, to ascertain a PND risk-score or a PVCI diagnostic score. In aspects, an at-risk susceptibility of the individual to perioperative neurocognitive disorder is ascertained by comparing the PND risk-score to pre-defined threshold criteria. In other aspects, identification or confirmation of a diagnosis in the individual of a post-viral cognitive impairment is ascertained by comparing the PVCI diagnostic-score to pre-defined threshold criteria. The disclosed systems, methods, and uses can further comprise determining a weighed score for the value of each of the one or more risk-related factors as well as further provide a treatment recommendation based on an individual’s at-risk susceptibility.

This is a continuation of International Application No. PCT/EP2022/050916, filed Jan. 17, 2022, which claims the benefit of priority and is entitled to the filing date pursuant to 35 U.S.C. § 119(e) of U.S. Provisional Pat. Application 63/137,802, filed Jan. 15, 2021, both of which are hereby incorporated by reference in their entireties.

With the advance of modern medicine, increasing numbers of older individuals are undergoing medical procedures for the treatment of disease or disorders with the primary goal being the improvement of function and quality of life. Many of these procedures involve surgery and anesthesia. In Western countries, for example, approximately 37% of all surgical procedures are performed on patients 65 years of age or older. Unfortunately, impairments in cognitive ability are the most common complications experienced by older individuals undergoing such surgical procedures.

Clinical studies since the 1980s have consistently documented decline in cognitive function in elderly individuals after anesthesia and surgery, even in the absence of symptoms. This work has identified increasing age, lower premorbid intelligence quotient, and fewer years of education as risk factors, and postoperative delirium as the most common complication. However the spectrum and complexity of cognitive impairment observed after anaesthesia and surgery, encompassing acute delirium to chronic postoperative cognitive dysfunction, has led to the classified of such cognitive change as a perioperative neurocognitive disorder.

Individuals suffering from a perioperative neurocognitive disorder are at risk for significant complications including dementia and even death. Sadly, the pathophysiology of a perioperative neurocognitive disorder and causal relationship between surgical procedures and the onset of this disorder still remain unknown. In addition, although risk factors are recognized, there is still no accurate or standardized means of identifying individuals at risk for a perioperative neurocognitive disorder. As a result an individual with a perioperative neurocognitive disorder is not routinely assessed or treated. As such, a perioperative neurocognitive disorder presents a major challenge to the rapidly growing aging population that negatively affect cognitive domains such as memory, attention, and concentration after surgery.

It has recently been observed that individuals suffering from viral infection are at risk for significant for a post-viral cognitive impairment. Studies among individuals of various ages have indicated an association between decreased cognitive functioning and exposure to neurotropic infectious agents including Herpes Simplex Virus Type 1 (HSV-1), Herpes Simplex Virus Type 2 (HSV-2), Cytomegalovirus (CMV), and Toxoplasma gondii (TOX). Increasing evidence in now revealing that infection with Sars-CoV-2 causes neurological deficits in a substantial proportion of affected patients. There are at least four possible pathogenic mechanisms where a viral infection can directly affect the brain and result in cognitive impairment as well as mood disorders: 1) direct viral encephalitis; 2) systemic inflammation; 3) peripheral organ dysfunction (liver, kidney, lung); and 4) cerebrovascular changes. In most cases, cognitive impairment due to a viral infection can arise from one or a combination of the above.

The present specification recognizes this need and provides methods and systems for identifying an individual at-risk for a perioperative neurocognitive disorder and diagnosing an individual suffering from a post-viral cognitive impairment, and methods and uses for treating an individual suffering from these disorders.

### SUMMARY

Aspects of the present specification disclose systems, methods, and uses of identifying an individual at risk of a perioperative neurocognitive disorder (PND) prior to a surgery. In some aspects, the disclosed systems, methods, and uses comprise comparing an episodic memory score, a working memory score, or both for an individual using a risk stratification matrix to determine a PND risk-score that ascertains an at-risk susceptibility of the individual to a perioperative neurocognitive disorder. In some aspects, the disclosed systems, methods, and uses comprise determining a standard deviation deficit by comparing to a normative mean to ascertain an episodic memory criterion value, a working memory criterion value, or a combined episodic memory and working memory criterion value for the individual; determining a PND risk-score based on the standard deviation deficit for the episodic memory score, the working memory score, or the combined episodic memory and working memory score; and determining an at-risk susceptibility of the individual to perioperative neurocognitive disorder by comparing the PND risk-score to pre-defined threshold criteria. In some aspects, the disclosed systems, methods, and uses comprise comparing an episodic memory score, a working memory score, or both for an individual in conjunction with one or more additional risk-related factors from the individual using a risk stratification matrix to determine a PND risk-score that ascertains an at-risk susceptibility of the individual to a perioperative neurocognitive disorder. In some aspects, the disclosed systems, methods, and uses comprise determining a standard deviation deficit by comparing to a normative mean to ascertain an episodic memory criterion value, a working memory criterion value, or both for the individual; determining the criterion value of one or more risk-related factors for the perioperative neurocognitive disorder in the individual; determining a PND risk-score based on the standard deviation deficit for the episodic memory score, the working memory score, or both in conjunction with the criterion value for each of the one or more risk-related factors; and determining an at-risk susceptibility of the individual to perioperative neurocognitive disorder by comparing the PND risk-score to pre-defined threshold criteria. The disclosed systems, methods, and uses can further comprise determining a weighed score for the value of an episodic memory score, a working memory score, or a combined episodic memory and working memory score as well as each of the one or more risk-related factors as well as further provide a treatment recommendation based on an individual’s at-risk susceptibility.

Other aspects of the present specification disclose systems, methods, and uses of diagnosing an individual suffering with cognitive impairment following a viral infection, i.e., a post-viral cognitive impairment (PVCI). In some aspects, the disclosed systems, methods, and uses comprise comparing an episodic memory score, a working memory score, or both for an individual in conjunction using a risk stratification matrix to identify or confirm a diagnosis of a PVCI in the individual. In some aspects, the disclosed systems, methods, and uses comprise determining a standard deviation deficit by comparing to a normative mean to ascertain an episodic memory criterion value, a working memory criterion value, or a combined episodic memory and working memory criterion value for the individual; determining a PVCI diagnostic-score based on the standard deviation deficit for the episodic memory score, the working memory score, or the combined episodic memory and working memory score; and determining an at-risk susceptibility of the individual to perioperative neurocognitive disorder by comparing the PVCI diagnostic-score to pre-defined threshold criteria. In some aspects, the disclosed systems, methods, and uses comprise comparing an episodic memory score, a working memory score, or both for an individual in conjunction with one or more additional risk-related factors from the individual using a risk stratification matrix to identify or confirm a diagnosis of a PVCI in the individual. In some aspects, the disclosed systems, methods, and uses comprise determining a standard deviation deficit by comparing to a normative mean to ascertain an episodic memory criterion value, a working memory criterion value, or both for the individual; determining the criterion value of one or more risk-related factors for the post-viral cognitive impairment in the individual; determining a PVCI diagnostic-score based on the standard deviation deficit for the episodic memory score, the working memory score, or both in conjunction with the criterion value for each of the one or more risk-related factors; and determining an at-risk susceptibility of the individual to perioperative neurocognitive disorder by comparing the PVCI diagnostic-score to pre-defined threshold criteria. The disclosed systems, methods, and uses can further comprise determining a weighed score for the value of an episodic memory score, a working memory score, or a combined episodic memory and working memory score as well as each of the one or more risk-related factors as well as further provide a treatment recommendation based on an individual’s diagnosis.

Other aspects of the present specification disclose a method of treating a perioperative neurocognitive disorder or a post-viral cognitive impairment by administering a composition comprising one or more fibrates. Other aspects of the present specification disclose a composition comprising one or more fibrates for use in treating a perioperative neurocognitive disorder or a post-viral cognitive impairment. Other aspects of the present specification disclose use of a composition comprising one or more fibrates in the treatment of a perioperative neurocognitive disorder or a post-viral cognitive impairment. Other aspects of the present specification disclose use of one or more fibrates in the manufacture of a medicament for the treatment of a perioperative neurocognitive disorder or a post-viral cognitive impairment. Non-limiting examples of a fibrate include bezafibrate, ciprofibrate, clinofibrate, clofibrate, clofibride, fenofibrate, fenofibric acid, gemfibrozil, ronifibrate, simfibrate, or any combination thereof. A perioperative neurocognitive disorder includes, without limitation, a delayed postoperative neurocognitive recovery (or early post-operative cognitive dysfunction), a mild postoperative neurocognitive disorder (or long-term post-operative cognitive dysfunction), a major postoperative neurocognitive disorder (or long-term post-operative cognitive dysfunction), a mild neurocognitive disorder (or long-term post-operative cognitive dysfunction), or a major neurocognitive disorder (or long-term post-operative cognitive dysfunction).

### DETAILED DESCRIPTION

Aspects of the present specification disclose systems, methods and uses of prospectively identifying an individual at risk of a perioperative neurocognitive disorder (PND). In some embodiments, a disclosed method and system prospectively identifies an individual at risk of a PND prior to a surgery or other operation. In some embodiments, the disclosed systems, methods and uses employ an episodic memory score for an individual to determine a PND risk-score that ascertains an at-risk susceptibility of the individual to a perioperative neurocognitive disorder. In some embodiments, the disclosed systems, methods and uses employ an episodic memory score for an individual in conjunction with one or more additional risk-related factors from the individual to determine a PND risk-score that ascertains an at-risk susceptibility of the individual to a perioperative neurocognitive disorder. In some embodiments, the disclosed systems, methods and uses employ a working memory score for an individual to determine a PND risk-score that ascertains an at-risk susceptibility of the individual to a perioperative neurocognitive disorder. In some embodiments, the disclosed systems, methods and uses employ a working memory score for an individual in conjunction with one or more additional risk-related factors from the individual to determine a PND risk-score that ascertains an at-risk susceptibility of the individual to a perioperative neurocognitive disorder. In some embodiments, the disclosed systems, methods and uses employ both an episodic memory score and a working memory score for an individual in conjunction with one or more additional risk-related factors from the individual to determine a PND risk-score that ascertains an at-risk susceptibility of the individual to a perioperative neurocognitive disorder. In some embodiments, the disclosed systems, methods and uses employ both an episodic memory score and a working memory score for an individual to determine a PND risk-score that ascertains an at-risk susceptibility of the individual to a perioperative neurocognitive disorder. The disclosed systems, methods, and uses can further comprise determining a weighed score for the value of an episodic memory score, a working memory score, or a combined episodic memory and working memory score as well as each of the one or more risk-related factors as well as further provide a treatment recommendation based on an individual’s at-risk susceptibility.

Aspects of the present specification disclose a perioperative neurocognitive disorder (PND). PND defines a syndrome characterized by the progressive deterioration of sensory and cognitive function during the preoperative or postoperative surgical period. Incidence rates of 20-80% have been reported and although prevalent, PNDs are not exclusively observed in elderly individuals. PNDs may have an acute phase of delirium; and/or a more chronic phase of cognitive impairment which tends to persist over time. Current recommendations regarding the classification of a PND are based on an objective assessment of impairment or decline in one or more domains compared with controls or normative data, a subjective complaint (patient or informant), and an assessment of activities of daily living (ADL) using the Diagnostic and Statistical Manual of Mental Disorders, Fifth Edition (DSM-5). See Evered, et al., Br. J. Anaesth. 121(5); 1005-1012 (2018; and Mahanna-Gabrielli, et al., Br. J. Anaesth. 123(4); 464-478 (2019), each of which is hereby incorporated by reference in its entirety.

Generally, PND defines seven broad categories: 1) preoperative neurocognitive disorder; 2) postoperative delirium; 3) delayed postoperative neurocognitive recovery; 4) mild postoperative neurocognitive disorder; 5) major postoperative neurocognitive disorder; 6) mild neurocognitive disorder; and 7) major neurocognitive disorder. A preoperative neurocognitive disorder includes any cognitive impairment or decline diagnosed preoperatively in an individual. A postoperative delirium includes any acute cognitive impairment or decline diagnosed 1 day and up to 7 days postoperatively or until discharge (whichever event occurs first) where the individual exhibits acute and fluctuating disturbances in attention and awareness. A delayed neurological recovery includes any cognitive impairment or decline diagnosed 1 day and up to 30 days postoperatively where the individual exhibits mild cognitive impairment (MCI) based on an objective impairment of 1-2 standard deviations below control/norm, a subjective complaint and ADL is not impaired or dementia based on an objective impairment of greater than two standard deviations below control/norm, a subjective complaint and ADL is impaired. A delayed neurological recovery is referred to in older literature as an early post-operative cognitive dysfunction.

A mild postoperative neurocognitive disorder includes any cognitive impairment or decline diagnosed 30 days and up to 12 months postoperatively where the individual exhibits mild cognitive impairment (MCI) based on an objective impairment of 1-2 standard deviations below control/norm, a subjective complaint and ADL is not impaired. A major postoperative neurocognitive disorder includes any cognitive impairment or decline diagnosed 30 days and up to 12 months postoperatively where the individual exhibits dementia based on an objective impairment of greater than two standard deviations below control/norm, a subjective complaint and ADL is impaired. A mild neurocognitive disorder includes any cognitive impairment or decline diagnosed over 12 months postoperatively and exhibiting mild cognitive impairment (MCI) based on an objective impairment of 1-2 standard deviations below control/norm, a subjective complaint and ADL is not impaired. A major neurocognitive disorder includes any cognitive impairment or decline diagnosed over 12 months postoperatively and exhibiting dementia based on an objective impairment of greater than two standard deviations below control/norm, a subjective complaint and ADL is impaired. A mild postoperative neurocognitive disorder, a major postoperative neurocognitive disorder, a mild neurocognitive disorder, a major neurocognitive disorder is generally grouped together in older literature and are referred to as a long-term post-operative cognitive dysfunction.

Aspects of the present specification disclose systems, methods and uses of identifying or confirming a diagnosis of a post-viral cognitive impairment (PVCI) in an individual. In some embodiments, the disclosed systems, methods and uses employ an episodic memory score for an individual, optionally in conjunction with one or more additional risk-related factors from the individual, to identify or confirm a diagnosis of a PVCI in the individual. In other embodiments, the disclosed systems, methods and uses employ a working memory score for an individual. optionally in conjunction with one or more additional risk-related factors from the individual, to identify or confirm a diagnosis of a PVCI in the individual. In yet other embodiments, the disclosed systems, methods and uses employ both an episodic memory score and a working memory score for an individual, optionally in conjunction with one or more additional risk-related factors from the individual, to identify or confirm a diagnosis of a PVCI in the individual. The disclosed systems, methods, and uses can further comprise determining a weighed score for the value of an episodic memory score, a working memory score, or a combined episodic memory and working memory score as well as each of the one or more risk-related factors as well as further provide a treatment recommendation based on an individual’s diagnosis.

Aspects of the present specification disclose a post-viral cognitive impairment (PVCI). PVCI defines a syndrome characterised by the deterioration of cognitive function during the presence of a viral infection or following the recovery of a viral infection. Incident rates of 20-80% have been reported and although prevalent, PCVI is not exclusively observed in elderly individuals. In most cases, PVCI represents a chronic phase of cognitive impairment which tends to persist or deteriorate over time. There are no current recommendations regarding a means to diagnose or identify PVCI and thus the present specification is based on an objective assessment of impairment or decline in one or more cognitive domains compared to normative data, and thus in accordance with the DSM-5 diagnosis for a mild neurocognitive disorder.

Part of the flexibility that humans show during behavior arises from their access to multiple memory systems that provide a variety of information. Two commonly studied memory systems are episodic memory and working memory. Episodic memory is a longer lasting memory that allows one to recall and re-experience personal events whereas working memory has been defined as memory that is active and relevant only for a short period of time. The hippocampal region of the brain is well-established for its role in memory function and is particularly vulnerable to the detrimental effects of inflammation caused by surgery or viral infection, and as such cognitive impairment of domains closely associated with hippocampal function are most likely to associated with a PND and/or PVCI. As both episodic and working memory are each a hippocampal function, the present methods and systems take advantage of the fact that poor pre-surgical episodic and/or working memory will be a risk factor for a PND and poor post-viral infection episodic and/or working memory will be a determining factor for a PVCI.

Aspects of the present specification disclose the step of analyzing episodic memory of an individual to generate an episodic memory score. In some embodiments, an episodic memory score of an individual is determined by comparing performance of an episodic memory test (EMT) to a control episodic memory score. In aspects of these embodiments, a control episodic memory score is obtained from normative data compiled from the general population. A score resulting in a negative value would indicate that an individual’s performance was worse than the normative mean while a score resulting in a positive value would indicate that an individual’s performance was better than the normative mean. In aspects of these embodiments an episodic memory score is determined by the following formula: Z_{EMT} = - (EMT_{individual} _{score} - EMTₙₒᵣₘₐₜᵢᵥₑ ₘₑₐₙ) / EMTₙₒᵣₘₐₜᵢᵥₑ _{SD}.

In some embodiments, an episodic memory in an individual is determined using an episodic memory test. Non-limiting examples of an episodic memory test include a list learning test, a recognition memory test and a paired associate learning test.

In some embodiments, determining episodic memory is accomplished using a list learning test. List learning evaluates a wide diversity of functions including short-term auditory-verbal memory, rate of learning, learning strategies, retroactive, and proactive interference, presence of confabulation of confusion in memory processes, retention of information, and differences between learning and retrieval. Tests designed to ascertain list learning typically comprise a pre-test phase including multiple trials where in each trial an individual is presented a list of unrelated words one by one and then asked to recall as many words possible (free recall) and a test phase where an individual is presented a different list of unrelated words one by one and asked to recall as many words possible (interference list) but then at a subsequent time asked to repeat the original list of words presented in the pre-test phase (delayed recall). One particular example of a list learning test is the Rey Auditory Verbal Learning Test (RAVLT). RAVLT includes a pre-test phase, where an individual is presented a Word List A of 15 unrelated words one by one and then asked to recall as many words as possible (free recall), this process being repeated five times, and then a test phase, where the individual is presented a different list of 15 unrelated words (Word List B) one by one and then asked to recall as many words as possible from Word List B (interference list) and then 20-30 minutes later the individual is asked to recall as many of the 15 words from Word List A (delayed recall). Two other examples of a list learning test are the Word List task from the Consortium to Establish a Registry for Alzheimer’s disease Neuropsychological Assessment Battery (CERAD-NAB) and the California Verbal Learning Test (CVLT).

In some embodiments, determining episodic memory is accomplished using a recognition memory test (RMT). Recognition memory evaluates verbal memory and new learning by assessing the ability to encode and subsequently retrieve verbal information, with recall tapping into fronto-temporal networks and recognition assessing hippocampal areas. Tests designed to ascertain recognition memory typically comprise a pre-test phase where an individual is shown a sequence of words one by one and the then asked to recall as many words as possible, the individual being scored as to which ones the individual remembered, and a test phase where the individual is presented with two words, one from the original list and one distractor and is then asked to choose which word was seen before, in a 2-force choice paradigm, immediately and then again at a subsequent time. One particular example of a recognition memory test is the Cambridge Neuropsychological Test Automated Battery (CANTAB) Verbal Recognition Memory (CANTAB VRM). CANTAB VRM includes a pre-test phase where an individual is presented 18 words displayed on-screen and then asked to recall as many as possible (free recall), and a test phase where an individual is presented 36 words (comprising 18 words from the pre-test list and 18 distractor words) and is asked to answer “yes” or “no” as to whether the individual saw the word previously. After a 20-minute delay period, another recognition test is then carried out, this time using the same 18 words from the pre-test list but with a new set of 18 distractor words.

In some embodiments, determining episodic memory is accomplished using a paired associates learning (PAL) test. Paired associates learning is the ability to remember an association of one stimulus to another. Tests designed to ascertain paired associate learning typically comprise a pre-test phase where an individual is exposed to a list of arbitrarily assigned pairs of stimuli, e.g., verbal or visual stimuli, and a test phase where one of the paired stimuli is presented to which the individual must recall the second associated stimulus. One particular example of a paired associates learning test is the Cambridge Neuropsychological Test Automated Battery (CANTAB) Paired Associates Learning Task (CANTAB PAL). CANTAB PAL displays boxes on a screen that open one by one in a randomised order to reveal patterns hidden inside. The patterns are then displayed in the middle of the screen, one at a time, and the participant must touch the box where the pattern was originally located. If the participant makes an error, the patterns are re-presented to remind the participant of their locations. As the test progresses the stages become more difficult as the number of patterns to be remembered increases. Another example is the Weschler Memory Scale: Verbal Paired Associates where the task is to learn eight unrelated word pairs across four study test trials, followed after 30 minutes by a delayed recall test and a recognition test.

Aspects of the present specification disclose the step of analyzing working memory of an individual to generate a working memory score. In some embodiments, a working memory score of an individual is determined by comparing performance of a working memory test (WMT) to a control working memory score. In aspects of these embodiments, a control working memory score is obtained from normative data compiled from the general population. A score resulting in a negative value would indicate that an individual’s performance was worse than the normative mean while a score resulting in a positive value would indicate that an individual’s performance was better than the normative mean. In aspects of these embodiments a working memory score is determined by the following formula: Z_{WMT} = - (WMT_{individual} _{score}-WMTₙₒᵣₘₐₜᵢᵥₑ ₘₑₐₙ) / WMTₙₒᵣₘₐₜᵢᵥₑ _{SD}.

In some embodiments, a working memory in an individual is determined using a working memory test. Non-limiting examples of a working memory test include a spatial working memory test, a spatial span test, a digit sequencing test, and a N-back test.

In some embodiments, determining working memory is accomplished using a spatial working memory (SWM) test. Spatial working memory tests designed to ascertain working memory typically requires retention and manipulation of visuospatial information. This test has notable executive function demands, and measures strategy use as well as errors. One particular example of a spatial working memory test requires a participant to search for tokens hidden in boxes. The participant must touch a box to open the box to reveal either a yellow token or an empty box. Once the participant has found a yellow token, they must touch the outline of the right-hand side of a screen to a computer device to ‘store’ it. The participant must then continue searching through the boxes until all of the tokens have been found. The key test instruction is that the computer will never hide a token in the same box, so once a token is found in a box the participant should not return to that box to look for another token. The colour and position of the boxes used are changed from trial to trial to discourage the use of stereotyped search strategies. As the test progresses, the number of tokens to find increases.

In some embodiments, determining working memory is accomplished using a spatial span test. Spatial span tests designed to ascertain working memory typically by assessing visuospatial working memory capacity, the component of working memory that allows you to temporarily hold and manipulate information about places. Spatial span tests typically comprise presenting an individual a series of flashing stimuli one by one and then after the presentation is completed asking the individual to recall the stimuli in the same order presented. Non-limiting examples of a spatial span test include the Cambridge Neuropsychological Test Automated Battery (CANTAB) Spatial Span (CANTAB SSP) test and the Wechsler Memory Scale: Spatial Span test.

In some embodiments, determining working memory is accomplished using a digit sequencing test. Digit sequencing tests designed to ascertain working memory typically by assessing verbal working memory capacity. Digit sequencing tests typically comprise presenting an individual a series of numbers of increasing complexity where the individual is asked to repeat each number immediately after presentation. The test continues until the individual cannot remember the complete sequence of the number or until the number is repeated incorrectly.

In some embodiments, determining working memory is accomplished using a N-back test. N-back tests designed to ascertain working memory typically N-back tests typically comprise presenting an individual with a sequence of stimuli one by one and then for each stimulus, the individual is asked to recall the stimulus previously presented N presentations ago where N can be 1, 2, 3, etc., where the higher the number N is, the more difficult the task. In one example, an individual is presented a series of numbers one by one and then asked to recall the number that was presented two numbers ago.

Aspects of the present specification disclose the step of analyzing both episodic memory and working memory of an individual to generate a combined episodic memory and working memory score. In some embodiments, a combined episodic memory and working memory score of an individual is determined by comparing performance of both an episodic memory test and a working memory test as disclosed herein to a control episodic memory and working memory scores. In aspects of these embodiments, a control combined episodic memory and working memory score is obtained from normative data compiled from the general population. A score resulting in a negative value would indicate that an individual’s performance was worse than the normative mean while a score resulting in a positive value would indicate that an individual’s performance was better than the normative mean.

In some embodiments, a combined episodic memory and working memory score is generated by first separately determining an episodic memory of an individual to generate an episodic memory score as disclosed herein and a working memory of an individual to generate a working memory score as disclosed herein. Once determined, these two scores are then added together to generate a combined episodic memory and working memory score. In aspects of these embodiments a combined episodic memory and working memory score is determined by the following formulas: i) determine an episodic memory score use the following formula: Z_{EMT} = - (EMT_{individual} score - EMTₙₒᵣₘₐₜᵢᵥₑ mean) / EMTₙₒᵣₘₐₜᵢᵥₑ _{SD}; 2) determine a working memory score use the following formula: Z_{WMT}= - (WMT_{individual} _{score} - WMTₙₒᵣₘₐₜᵢᵥₑ ₘₑₐₙ) / WMT_{normative SD}; and 3) determine a combined episodic memory and working memory score by adding the two scores and taking the average using the following formula: Combined risk score Z_{CMT} = (Z_{EMT} + Z_{WMT}) / 2.

The spectrum of abilities referred to as cognition is diverse, including learning and memory, verbal abilities, perception, attention, executive functions, and abstract thinking. Cognitive assessment evaluations should objectively assess specific cognitive domains. In some embodiments, a cognitive assessment evaluation is conducted to obtain both a pre-operative baseline of cognition for an individual and a post-operative determination of cognition.

Aspects of the present specification disclose the step of determining risk-related factors of an individual. Typically this risk-related factors includes information about an individual’s personal and medical history as well as cognitive assessment evaluation. Information from an individual’s condition and medical history includes, without limitation, age, gender, height, weight, body mass index (BMI), education level, intelligence quotient, emotional quotient, and other biometric information, physical exam results, information regarding prior viral infection(s), information of cognitive concerns from individual and family members, information regarding changes in behavioral, mood or thinking such as, e.g., hallucinations, delusions, personality changes, apathy, depression, anxiety, disorientation, and confusion, information regarding changes in physical health, such as, e.g., difficulties with walking, balance, speech, and coordination, tremor and stiffness, information regarding substance abuse and withdrawal, whether this is an initial assessment or a subsequent assessment of an individual, whether the individual is currently experiencing symptoms of cognitive impairment, whether the individual has a history of cognitive impairment, whether the individual has been previously diagnosed with a cognitive impairment, whether the individual is currently experiencing symptoms of an inflammatory related disease, infection or physical illness, whether the individual has a history of an inflammatory related disease, infection or physical illness, whether the individual has been previously diagnosed with an inflammatory related disease, infection or physical illness, whether the individual is currently taking anticholinergic medication. Additionally, perioperative information, such as surgery duration, surgery type, anesthetic management, and intraoperative physiology may also be collected. Also, any or additional factors that may act to decrease cognitive reserve and be a risk for post-operative cognitive change.

Aspects of the present specification disclose the step of determining one or more risk-related factors to generate a criterion value of each of the one or more risk-related factors. In some embodiments, a risk-related factor score of an individual is determined by comparing the value of a risk-related factor to a control risk-related factor score. In aspects of these embodiments, a risk-related factor criterion value is obtained by comparing the value of a risk-related factor to normative data compiled from the general population and determining whether the risk-related score of the individual falls within a range of acceptable values indicating that the individual is not at-risk or whether the risk-related score of the individual falls outside the acceptable range of values indicating that the individual is at-risk. In aspects of these embodiments, when determining an inflammation criterion value, a C-Reactive Protein (CRP) blood level of less than 10 mg/L is indicative of a not at-risk inflammation criterion value while a CRP blood level of 10 mg/L or more is indicative of an at-risk inflammation criterion value. In aspects of these embodiments, when determining an age criterion value, less than 60 years old is indicative of a not at-risk age criterion value while a 60 years old or more is indicative of an at-risk age criterion value. In aspects of these embodiments, when determining a BMI criterion value, a value of less than 30 is indicative of a not at-risk BMI criterion value while a value of 30 or more is indicative of an at-risk BMI criterion value. In aspects of these embodiments, when determining an education criterion value, a value of more than 12 years of formal education is indicative of a not at-risk education criterion value while a value of 12 years of formal education or less is indicative of an at-risk education criterion value. In aspects of these embodiments, when determining an medication criterion value, no use of at-risk medication is indicative of a not at-risk medication criterion value while use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication is indicative of an at-risk medication criterion value. In aspects of these embodiments, when determining a disease diagnosis or history criterion value, a lack of any diagnosis or history of an at-risk disease is indicative of a not at-risk disease diagnosis or history criterion value while the presence of a psychiatric disorder, a neurological disorder, a diabetes, a metabolic syndrome, an autoimmune disease, a heart disease, a vascular disease, a stroke, an infection, an injury, an asthma, or alcohol abuse is indicative of an at-risk disease diagnosis or history criterion value.

Aspects of the present specification disclose the step of determining a PND risk-score that ascertains an at-risk susceptibility of an individual for a PND. In some embodiments, a PND risk-score is based on a standard deviation deficit of an individual’s episodic memory score from a normative mean. In some embodiments, a PND risk-score is based on a standard deviation deficit of an individual’s episodic memory score from a normative mean in conjunction with a criterion value one or more risk-related factors. In some embodiments, a PND risk-score is based on a standard deviation deficit of an individual’s working memory score from a normative mean. In some embodiments, a PND risk-score is based on a standard deviation deficit of an individual’s working memory score from a normative mean in conjunction with a criterion value one or more risk-related factors. In some embodiments, a PND risk-score is based on a standard deviation deficit of an individual’s combined episodic memory and working memory score from a normative mean. In some embodiments, a PND risk-score is based on a standard deviation deficit of an individual’s combined episodic memory and working memory score from a normative mean in conjunction with a criterion value one or more risk-related factors.

In some embodiments, an at-risk susceptibility of an individual for a PND uses a risk stratification matrix that employs pre-defined threshold criteria values to an episodic memory score, a working memory score and each additional risk-related factor by which an individual’s PND risk score is determined and an at-risk susceptibility assessed. In some embodiments, each pre-defined threshold criteria values to an episodic memory score, a working memory score and each additional risk-related factor disclosed herein is determined using a normative mean. A normative mean is the average value of data or other information (norms) taken from a population of interest that establishes a baseline distribution of results for that particular population and includes, without limitation, an average value of an episodic memory score, a working memory score, as well as the other risk-related factors disclosed herein, taken from a healthy population cohort, or an average value based on previous episodic memory scores, working memory scores, and/or the other risk-related factors disclosed herein obtained from the individual undergoing a method or use disclosed herein.

In some embodiments, pre-defined threshold criteria values identifying an individual at risk for a PND is determined by an episodic memory criterion value. In aspects of these embodiments, an episodic memory criterion value includes a score of greater than or equal to a standard deviation of one below a normative mean (Zscore conversion is equal to or less than -1). In some embodiments, pre-defined threshold criteria values identifying an individual at risk for a PND is determined by a working memory criterion value. In aspects of these embodiments, a working memory criterion value includes a score of greater than or equal to a standard deviation of one below a normative mean (Zscore conversion is equal to or less than -1). In some embodiments, pre-defined threshold criteria values identifying an individual at risk for a PND is determined by a combined episodic memory and a working memory criterion value. In aspects of these embodiments, a combined episodic memory and working memory criterion value includes a score of greater than or equal to a standard deviation of one below a normative mean (Zscore conversion is equal to or less than -1).

In some embodiments, pre-defined threshold criteria values identifying an individual at risk for a PND is determined by an episodic memory criterion value and the presence of an inflammation criterion value. In aspects of these embodiments, an episodic memory criterion value includes a score of greater than or equal to a standard deviation of one below a normative mean (Zscore conversion is equal to or less than -1) and an inflammation criterion value includes the presence of a CRP blood level of 10 mg/L or more. In some embodiments, pre-defined threshold criteria values identifying an individual at risk for a PND is determined by a working memory criterion value and the presence of an inflammation criterion value. In aspects of these embodiments, a working memory criterion value includes a score of greater than or equal to a standard deviation of one below a normative mean (Zscore conversion is equal to or less than -1) and an inflammation criterion value includes the presence of a CRP blood level of 10 mg/L or more. In some embodiments, pre-defined threshold criteria values identifying an individual at risk for a PND is determined by a combined episodic memory and working memory criterion value, and the presence of an inflammation criterion value. In aspects of these embodiments, a combined episodic memory and working memory criterion value includes a score of greater than or equal to a standard deviation of one below a normative mean (Zscore conversion is equal to or less than -1), and an inflammation criterion value includes the presence of a CRP blood level of 10 mg/L or more.

In some embodiments, pre-defined threshold criteria values identifying an individual at risk for a PND is determined by an episodic memory criterion value and the presence of two or more factors with one factor being an age criterion value, an education criterion value, or a BMI criterion value, and the other factor being at least one of a diagnosis or history criterion value or a medication use criterion value. In aspects of these embodiments, an episodic memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by a working memory criterion value and the presence of two or more factors with one factor being an age criterion value, an education criterion value, or a BMI criterion value, and the other factor being at least one of a diagnosis or history criterion value or a medication use criterion value. In aspects of these embodiments, a working memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by a combined episodic memory and working memory criterion value, and the presence of two or more factors with one factor being an age criterion value, an education criterion value, or a BMI criterion value, and the other factor being at least one of a diagnosis or history criterion value or a medication use criterion value. In aspects of these embodiments, a combined episodic memory and working memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by an episodic memory criterion value and the presence of two or more factors with one factor being an age criterion value, an education criterion value, or a BMI criterion value, and the other factor being medication use criterion value. In aspects of these embodiments, an episodic memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by a working memory criterion value and the presence of two or more factors with one factor being an age criterion value, an education criterion value, or a BMI criterion value, and the other factor being medication use criterion value. In aspects of these embodiments, a working memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by a combined episodic memory and working memory criterion value, and the presence of two or more factors with one factor being an age criterion value, an education criterion value, or a BMI criterion value, and the other factor being medication use criterion value. In aspects of these embodiments, a combined episodic memory and working memory criterion value includes a score of greater than or equal to a standard deviation of one below a normative mean (Zscore conversion is equal to or less than -1); an age criterion value includes 60 years old or more, an education criterion value includes 12 years of formal education or less, and a BMI criterion value includes a score of 30 or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying an individual at risk for a PND is determined by an episodic memory criterion value and the presence of two or more factors with one factor being an age criterion value and the other factor being at least one of a disease diagnosis or history criterion value. In aspects of these embodiments, an episodic memory criterion value includes a score of greater than or equal to a standard deviation of one below a normative mean (Zscore conversion is equal to or less than -1); an age criterion value includes 60 years old or more, and a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying an individual at risk for a PND is determined by a working memory criterion value and the presence of two or more factors with one factor being an age criterion value and the other factor being at least one of a disease diagnosis or history criterion value. In aspects of these embodiments, a working memory criterion value includes a score of greater than or equal to a standard deviation of one below a normative mean (Zscore conversion is equal to or less than -1); an age criterion value includes 60 years old or more, and a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying an individual at risk for a PND is determined by a combined episodic memory and working memory criterion value, and the presence of two or more factors with one factor being an age criterion value and the other factor being at least one of a disease diagnosis or history criterion value. In aspects of these embodiments, a combined episodic memory and working memory criterion value includes a score of greater than or equal to a standard deviation of one below a normative mean (Zscore conversion is equal to or less than -1); an age criterion value includes 60 years old or more, and a disease diagnosis or history criterion value includes the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying a person at risk for a PND is determined by an episodic memory criterion value and the presence of two or more factors with one factor being an age criterion value and the other factor being medication use criterion value. In aspects of these embodiments, an episodic memory criterion value includes a score of greater than or equal to a standard deviation of one below a normative mean (Zscore conversion is equal to or less than -1); an age criterion value includes 60 years old or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying a person at risk for a PND is determined by a working memory criterion value and the presence of two or more factors with one factor being an age criterion value and the other factor being medication use criterion value. In aspects of these embodiments, a working memory criterion value includes a score of greater than or equal to a standard deviation of one below a normative mean (Zscore conversion is equal to or less than -1); an age criterion value includes 60 years old or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying a person at risk for a PND is determined by a combined episodic memory and working memory criterion value, and the presence of two or more factors with one factor being an age criterion value and the other factor being medication use criterion value. In aspects of these embodiments, a combined episodic memory and working memory criterion value includes a score of greater than or equal to a standard deviation of one below a normative mean (Zscore conversion is equal to or less than -1); an age criterion value includes 60 years old or more; and a medication use criterion value includes the use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication.

In some embodiments, pre-defined threshold criteria values identifying a person at very high risk for a PND is determined by an episodic memory criterion value and poor criterion values of the risk-related factors for inflammation, age, BMI, education level, medication use, and disease diagnosis or history. In aspects of these embodiments, pre-defined threshold criteria values identifying a person at very high risk for a PND is determined by an episodic memory criterion value of greater than or equal to a standard deviation of one below a normative mean (Zscore conversion is equal to or less than -1); an inflammation criterion value indicative of high inflammation (e.g., a blood level of 10 mg/L or more of CRP); an age criterion value of 60 or more years; a BMI criterion value of 30 or more; an education criterion value of 12 years of formal education or less; an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication; and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying a person at very high risk for a PND is determined by a working memory criterion value and poor criterion values of the risk-related factors for inflammation, age, BMI, education level, medication use, and disease diagnosis or history. In aspects of these embodiments, pre-defined threshold criteria values identifying a person at very high risk for a PND is determined by a working memory criterion value of greater than or equal to a standard deviation of one below a normative mean (Zscore conversion is equal to or less than -1); an inflammation criterion value indicative of high inflammation (e.g., a blood level of 10 mg/L or more of CRP); an age criterion value of 60 or more years; a BMI criterion value of 30 or more; an education criterion value of 12 years of formal education or less; an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication; and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying a person at very high risk for a PND is determined by a combined episodic memory and working memory criterion value, and poor criterion values of the risk-related factors for inflammation, age, BMI, education level, medication use, and disease diagnosis or history. In aspects of these embodiments, pre-defined threshold criteria values identifying a person at very high risk for a PND is determined by a combined episodic memory and working memory criterion value includes a score of greater than or equal to a standard deviation of one below a normative mean (Zscore conversion is equal to or less than -1); an inflammation criterion value indicative of high inflammation (e.g., a blood level of 10 mg/L or more of CRP); an age criterion value of 60 or more years; a BMI criterion value of 30 or more; an education criterion value of 12 years of formal education or less; an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication; and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse.

In some embodiments, pre-defined threshold criteria values identifying a person at high risk for a PND is determined by an episodic memory criterion value. In aspects of these embodiments, pre-defined threshold criteria values identifying a person at high risk for a PND is determined by an episodic memory criterion value of greater than or equal to a standard deviation of one below a normative mean (Zscore conversion is equal to or less than -1).

In some embodiments, pre-defined threshold criteria values identifying a person at high risk for a PND is determined by a working memory criterion value. In aspects of these embodiments, pre-defined threshold criteria values identifying a person at high risk for a PND is determined by a working memory criterion value of greater than or equal to a standard deviation of one below a normative mean (Zscore conversion is equal to or less than -1).

In some embodiments, pre-defined threshold criteria values identifying a person at high risk for a PND is determined by an episodic memory criterion value and a working memory criterion value. In aspects of these embodiments, pre-defined threshold criteria values identifying a person at high risk for a PND is determined by a combined episodic memory and working memory criterion value includes a score of greater than or equal to a standard deviation of one below a normative mean (Zscore conversion is equal to or less than -1).

In some embodiments, pre-defined threshold criteria values identifying a person at high risk for a PND is determined by an episodic memory criterion value and an inflammation criterion value. The criterion values of the risk-related factors for age, BMI, education level, medication use, and disease diagnosis or history are all non-indicative. In aspects of these embodiments, pre-defined threshold criteria values identifying a person at high risk for a PND is determined by an episodic memory criterion value of greater than or equal to a standard deviation of one below a normative mean (Zscore conversion is equal to or less than -1); and an inflammation criterion value of a blood level of 10 mg/L or more of CRP. In other aspects of these embodiments, pre-defined threshold criteria values identifying a person at high risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at high risk for a PND is determined by a working memory criterion value and an inflammation criterion value. The criterion values of the risk-related factors for age, BMI, education level, medication use, and disease diagnosis or history are all non-indicative. In aspects of these embodiments, pre-defined threshold criteria values identifying a person at high risk for a PND is determined by a working memory criterion value of greater than or equal to a standard deviation of one below a normative mean (Zscore conversion is equal to or less than -1); and an inflammation criterion value of a blood level of 10 mg/L or more of CRP. In other aspects of these embodiments, pre-defined threshold criteria values identifying a person at high risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at high risk for a PND is determined by a combined episodic memory and working memory criterion value, and an inflammation criterion value. The criterion values of the risk-related factors for age, BMI, education level, medication use, and disease diagnosis or history are all non-indicative. In aspects of these embodiments, pre-defined threshold criteria values identifying a person at high risk for a PND is determined by a combined episodic memory and working memory criterion value includes a score of greater than or equal to a standard deviation of one below a normative mean (Zscore conversion is equal to or less than -1); and an inflammation criterion value of a blood level of 10 mg/L or more of CRP. In other aspects of these embodiments, pre-defined threshold criteria values identifying a person at high risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at high risk for a PND is determined by an episodic memory criterion value and an age criterion value. The criterion values of the risk-related factors for inflammation, BMI, education level, medication use, and disease diagnosis or history are all non-indicative. In aspects of these embodiments, pre-defined threshold criteria values identifying a person at high risk for a PND is determined by an episodic memory criterion value of greater than or equal to a standard deviation of one below a normative mean (Zscore conversion is equal to or less than -1); and an age criterion value of 60 or more years. In other aspects of these embodiments, pre-defined threshold criteria values identifying a person at high risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at high risk for a PND is determined by a working memory criterion value and an age criterion value. The criterion values of the risk-related factors for inflammation, BMI, education level, medication use, and disease diagnosis or history are all non-indicative. In aspects of these embodiments, pre-defined threshold criteria values identifying a person at high risk for a PND is determined by a working memory criterion value of greater than or equal to a standard deviation of one below a normative mean (Zscore conversion is equal to or less than -1); and an age criterion value of 60 or more years. In other aspects of these embodiments, pre-defined threshold criteria values identifying a person at high risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at high risk for a PND is determined by a combined episodic memory and working memory criterion value, and an age criterion value. The criterion values of the risk-related factors for inflammation, BMI, education level, medication use, and disease diagnosis or history are all non-indicative. In aspects of these embodiments, pre-defined threshold criteria values identifying a person at high risk for a PND is determined by a combined episodic memory and working memory criterion value includes a score of greater than or equal to a standard deviation of one below a normative mean (Zscore conversion is equal to or less than -1); and an age criterion value of 60 or more years. In other aspects of these embodiments, pre-defined threshold criteria values identifying a person at high risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by an episodic memory criterion value. In aspects of these embodiments, pre-defined threshold criteria values identifying a person at medium risk for a PND is determined by an episodic memory criterion value that is below the normative mean but less than one standard deviation below the normative mean (Zscore conversion is less than zero but greater than -1).

In some embodiments, pre-defined threshold criteria values identifying a person at medium risk for a PND is determined by a working memory criterion value. In aspects of these embodiments, pre-defined threshold criteria values identifying a person at medium risk for a PND is determined by a working memory criterion value that is below the normative mean but less than one standard deviation below the normative mean (Zscore conversion is less than zero but greater than -1).

In some embodiments, pre-defined threshold criteria values identifying a person at medium risk for a PND is determined by a combined episodic memory and working memory criterion value. In aspects of these embodiments, pre-defined threshold criteria values identifying a person at medium risk for a PND is determined by a combined episodic memory and working memory criterion value that is below the normative mean but less than one standard deviation below the normative mean (Zscore conversion is less than zero but greater than -1).

In some embodiments, pre-defined threshold criteria values identifying a person at medium risk for a PND is determined by an episodic memory criterion value and an inflammation criterion value. The criterion values of the risk-related factors for age, BMI, education level, medication use, and disease diagnosis or history are all non-indicative. In aspects of these embodiments, pre-defined threshold criteria values identifying a person at medium risk for a PND is determined by an episodic memory criterion value that is below the normative mean but less than one standard deviation below the normative mean (Zscore conversion is less than zero but greater than -1); and an inflammation criterion value of a blood level of 10 mg/L or more of CRP. In other aspects of these embodiments, pre-defined threshold criteria values identifying a person at medium risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by a working memory criterion value and an inflammation criterion value. The criterion values of the risk-related factors for age, BMI, education level, medication use, and disease diagnosis or history are all non-indicative. In aspects of these embodiments, pre-defined threshold criteria values identifying a person at medium risk for a PND is determined by a working memory criterion value that is below the normative mean but less than one standard deviation below the normative mean (Zscore conversion is less than zero but greater than -1); and an inflammation criterion value of a blood level of 10 mg/L or more of CRP. In other aspects of these embodiments, pre-defined threshold criteria values identifying a person at medium risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by a combined episodic memory and working memory criterion value, and an inflammation criterion value. The criterion values of the risk-related factors for age, BMI, education level, medication use, and disease diagnosis or history are all non-indicative. In aspects of these embodiments, pre-defined threshold criteria values identifying a person at medium risk for a PND is determined by a combined episodic memory and working memory criterion value that is below the normative mean but less than one standard deviation below the normative mean (Zscore conversion is less than zero but greater than -1); and an inflammation criterion value of a blood level of 10 mg/L or more of CRP. In other aspects of these embodiments, pre-defined threshold criteria values identifying a person at medium risk for a PND includes an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by an episodic memory criterion value and an age criterion value. The criterion values of the risk-related factors for inflammation, BMI, education level, medication use, and disease diagnosis or history are all non-indicative. In aspects of these embodiments, pre-defined threshold criteria values identifying a person at medium risk for a PND is determined by an episodic memory criterion value that is below the normative mean but less than one standard deviation below the normative mean (Zscore conversion is less than zero but greater than -1); and an age criterion value of 60 or more years. In other aspects of these embodiments, pre-defined threshold criteria values identifying a person at medium risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by a working memory criterion value and an age criterion value. The criterion values of the risk-related factors for inflammation, BMI, education level, medication use, and disease diagnosis or history are all non-indicative. In aspects of these embodiments, pre-defined threshold criteria values identifying a person at medium risk for a PND is determined by a working memory criterion value that is below the normative mean but less than one standard deviation below the normative mean (Zscore conversion is less than zero but greater than -1); and an age criterion value of 60 or more years. In other aspects of these embodiments, pre-defined threshold criteria values identifying a person at medium risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at medium risk for a PND is determined by a combined episodic memory and working memory criterion value, and an age criterion value. The criterion values of the risk-related factors for inflammation, BMI, education level, medication use, and disease diagnosis or history are all non-indicative. In aspects of these embodiments, pre-defined threshold criteria values identifying a person at medium risk for a PND is determined by a combined episodic memory and working memory criterion value that is below the normative mean but less than one standard deviation below the normative mean (Zscore conversion is less than zero but greater than -1); and an age criterion value of 60 or more years. In other aspects of these embodiments, pre-defined threshold criteria values identifying a person at medium risk for a PND includes an inflammation criterion value of a blood level of less than 10 mg/L of CRP; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at low risk for a PND is determined by an episodic memory criterion value. The criterion values of the risk-related factors for inflammation, age, BMI, education level, medication use, and disease diagnosis or history are all non-indicative. In aspects of these embodiments, pre-defined threshold criteria values identifying a person at low risk for a PND is determined by an episodic memory criterion value that is above the normative mean (Zscore conversion is equal to or greater than zero). In other aspects of these embodiments, pre-defined threshold criteria values identifying a person at low risk for a PND includes an inflammation criterion value of a blood level of less than 10 mg/L of CRP; an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at low risk for a PND is determined by a working memory criterion value. The criterion values of the risk-related factors for inflammation, age, BMI, education level, medication use, and disease diagnosis or history are all non-indicative. In aspects of these embodiments, pre-defined threshold criteria values identifying a person at low risk for a PND is determined by a working memory criterion value that is above the normative mean (Zscore conversion is equal to or greater than zero). In other aspects of these embodiments, pre-defined threshold criteria values identifying a person at low risk for a PND includes an inflammation criterion value of a blood level of less than 10 mg/L of CRP; an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In some embodiments, pre-defined threshold criteria values identifying a person at low risk for a PND is determined by a combined episodic and working memory criterion value. The criterion values of the risk-related factors for inflammation, age, BMI, education level, medication use, and disease diagnosis or history are all non-indicative. In aspects of these embodiments, pre-defined threshold criteria values identifying a person at low risk for a PND is determined by a combined episodic memory and working memory criterion value that is above the normative mean (Zscore conversion is equal to or greater than zero). In other aspects of these embodiments, pre-defined threshold criteria values identifying a person at low risk for a PND includes an inflammation criterion value of a blood level of less than 10 mg/L of CRP; an age criterion value of less than 60 years old; a BMI criterion value of less than 30; an education criterion value of more than 12 years of formal education; an medication use criterion value of no at-risk medication use; and a disease diagnosis or history criterion value of no at-risk disease diagnosis or history.

In addition, the risk stratification matrix can further stratify the PND risk-score of an individual to assess the at-risk severity of the individual. In some embodiments, a risk stratification matrix disclosed herein can apply a weighted score to each threshold criterion value assigned to an episodic memory score, a working memory score, a combined episodic and working memory score, each additional risk-related factor, or any combination thereof. In some embodiments, the weighted score assigned to each threshold criterion value by the risk stratification matrix is developed from data obtained from information compiled on a PND, such as, e.g., information obtained from observational studies, interventional studies, and clinical studies.

In some embodiments, stratification of the PND risk-score of an individual results in a recommendation that the individual undergo additional monitoring or treatment for a PND before and/or after surgery. In some embodiments, stratification of the PND risk-score of an individual results in the classification of a PND as a preoperative neurocognitive disorder, a postoperative delirium, a delayed postoperative neurocognitive recovery (or early post-operative cognitive dysfunction); a mild postoperative neurocognitive disorder (or long-term post-operative cognitive dysfunction); a major postoperative neurocognitive disorder (or long-term post-operative cognitive dysfunction), a mild neurocognitive disorder (or long-term post-operative cognitive dysfunction) or a major neurocognitive disorder (or long-term post-operative cognitive dysfunction).

If the PND risk-score of the individual meets certain predefined threshold criteria values, a risk stratification matrix disclosed herein will indicate that the individual is at risk of a PND. In addition, depending on which predefined threshold criteria values are meet, a risk stratification matrix disclosed herein can qualify the degree of at-risk susceptibility as well as the degree of cognitive impairment. In some embodiments, a risk stratification matrix disclosed herein can qualify the degree of at-risk susceptibility as very high risk for a PND, high risk for a PND, medium risk for a PND, low risk for a PND, or no risk for a PND. In some embodiments, a risk stratification matrix disclosed herein can qualify the degree of at-risk susceptibility as very high risk for a PND, high risk for a PND, medium risk for a PND, or low risk for a PND. In some embodiments, a risk stratification matrix disclosed herein can qualify the degree of at-risk susceptibility as high risk for a PND, medium risk for a PND, or low risk for a PND.

In some embodiments, a weighted score assigned to each threshold criterion value by the risk stratification matrix for an episodic memory score, a working memory score, a combined episodic and a working memory score, and each additional risk-related factor to ascertain a PND risk-score that determines an at-risk susceptibility of an individual for a PND is based on Table 1.

TABLE 1

Risk Stratification Matrix for PND Risk-Score Ascertainment and At-Risk Susceptibility

| Risk Factor | Individual’s Risk-Related Information | Weighted Score | PND Risk Score | Therapy Recommendation |
|---|---|---|---|---|
| Episodic Memory Score | ≥ 1SD deficit compared to normative mean (Zscore conversion ≤ -1) | 40-90% | Very High PND Risk | Treat with Fenofibrate |
| Inflammation Level | CRP > 10 mg/L | 20-90% | | |
| Age | ≥ 60 years | 20-90% | | |
| BMI | ≥ 30.0 | 5-20% | | |
| Education | ≤ 12 years | 5-20% | | |
| Medication Use (current)^{a} | ≥ 1 from risk list | 5-20% | | |
| Disease Diagnoses (current or history)^{b} | ≥ 1 from risk list | 5-20% | | |
| Working Memory Score | ≥ 1SD deficit compared to normative mean (Zscore conversion ≤ -1) | 40-90% | Very High PND Risk | Treat with Fenofibrate |
| Inflammation Level | CRP > 10 mg/L | 20-90% | | |
| Age | ≥ 60 years | 20-90% | | |
| BMI | ≥ 30.0 | 5-20% | | |
| Education | ≤ 12 years | 5-20% | | |
| Medication Use (current)^{a} | ≥ 1 from risk list | 5-20% | | |
| Disease Diagnoses (current or history)^{b} | ≥ 1 from risk list | 5-20% | | |
| Combined Episodic + Working Memory Score | ≥ 1SD deficit compared to normative mean (Zscore conversion ≤ -1) | 40-90% | Very High PND Risk | Treat with Fenofibrate |
| Inflammation Level | CRP > 10 mg/L | 20-90% | | |
| Age | ≥ 60 years | 20-90% | | |
| BMI | ≥ 30.0 | 5-20% | | |
| Education | ≤ 12 years | 5-20% | | |
| Medication Use (current)^{a} | ≥ 1 from risk list | 5-20% | | |
| Disease Diagnoses (current or history)^{b} | ≥ 1 from risk list | 5-20% | | |
| Episodic Memory Score | ≥ 1SD deficit compared to normative mean (Zscore conversion ≤ -1) | 100% | High PND Risk | Treat with Fenofibrate |
| Working Memory Score | ≥ 1SD deficit compared to normative mean (Zscore conversion ≤ -1) | 100% | High PND Risk | Treat with Fenofibrate |
| Combined Episodic + Working Memory Score | ≥ 1SD deficit compared to normative mean (Zscore conversion ≤ -1) | 100% | High PND Risk | Treat with Fenofibrate |
| Episodic Memory Score | ≥ 1SD deficit compared to normative mean (Zscore conversion -1) | 70-90% | High PND Risk | Treat with Fenofibrate |
| Inflammation Level | CRP > 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Working Memory Score | ≥ 1SD deficit compared to normative mean (Zscore conversion -1) | 70-90% | High PND Risk | Treat with Fenofibrate |
| Inflammation Level | CRP > 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Combined Episodic + Working Memory Score | ≥ 1SD deficit compared to normative mean (Zscore conversion -1) | 70-90% | High PND Risk | Treat with Fenofibrate |
| Inflammation Level | CRP > 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Episodic Memory Score | ≥ 1SD deficit compared to normative mean (Zscore conversion -1) | 60-90% | High PND Risk | Treat with Fenofibrate |
| Inflammation Level | CRP > 10 mg/L | 40-90% | | |
| Age | < 60 years | 0-20% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Working Memory Score | ≥ 1SD deficit compared to normative mean (Zscore conversion -1) | 60-90% | High PND Risk | Treat with Fenofibrate |
| Inflammation Level | CRP > 10 mg/L | 40-90% | | |
| Age | < 60 years | 0-20% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Combined Episodic + Working Memory Score | ≥ 1SD deficit compared to normative mean (Zscore conversion ≤ -1) | 60-90% | High PND Risk | Treat with Fenofibrate |
| Inflammation Level | CRP > 10 mg/L | 40-90% | | |
| Age | < 60 years | 0-20% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Episodic Memory Score | ≥ 1SD deficit compared to normative mean (Zscore conversion -1) | 70-90% | High PND Risk | Treat with Fenofibrate |
| Inflammation Level | CRP < 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Working Memory Score | ≥ 1SD deficit compared to normative mean (Zscore conversion -1) | 70-90% | High PND Risk | Treat with Fenofibrate |
| Inflammation Level | CRP < 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Combined Episodic + Working Memory Score | ≥ 1SD deficit compared to normative mean (Zscore conversion ≤ -1) | 70-90% | High PND Risk | Treat with Fenofibrate |
| Inflammation Level | CRP < 10 mg/L | 0-20% | | |
| Age | ≥ 60 years | 30-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Episodic Memory Score | Performance worse than normative mean (Zscore conversion = negative score) | 100% | Medium PND Risk | Treat with Fenofibrate |
| Working Memory Score | Performance worse than normative mean (Zscore conversion = negative score) | 100% | Medium PND Risk | Treat with Fenofibrate |
| Combined Episodic + Working Memory Score | Performance worse than normative mean (Zscore conversion = negative score) | 100% | Medium PND Risk | Treat with Fenofibrate |
| Episodic Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Working Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Combined Episodic + Working Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Episodic Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | < 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Working Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | < 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Combined Episodic + Working Memory Score | Performance worse than normative mean (Zscore conversion = negative score) | 70-90% | Medium PND Risk | Treat with Fenofibrate |
| Inflammation level | CRP > 10 mg/L | 0-20% | | |
| Age | < 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Episodic Memory Score | Performance worse than normative mean (Zscore conversion = negative score) | 70-90% | Medium PND Risk | Treat with Fenofibrate |
| Inflammation level | CRP < 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Working Memory Score | Performance worse than normative mean (Zscore conversion = negative score) | 70-90% | Medium PND Risk | Treat with Fenofibrate |
| Inflammation level | CRP < 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Combined Episodic + Working Memory Score | Performance worse than normative mean (Zscore conversion = negative score) | 70-90% | Medium PND Risk | Treat with Fenofibrate |
| Inflammation level | CRP < 10 mg/L | 0-20% | | |
| Age | ≥ 60 years | 0-20% | | |
| BMI | < 30.0 | 0-20% | | |
| Education | > 12 years | 0-20% | | |
| Medication Use (current)^{a} | = 0 from risk list | 15-50% | | |
| Disease Diagnoses (current or history)^{b} | = 0 from risk list | 15-50% | | |
| Episodic Memory Score | Performance better than normative mean (Zscore conversion = positive score) | 0-90% | Low PND Risk | Do not Treat / Treat with Standard of Care |
| Inflammation Level | CRP < 10 mg/L | 0-90% | | |
| Age | < 60 years | 0-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Working Memory Score | Performance better than normative mean (Zscore conversion = positive score) | 0-90% | Low PND Risk | Do not Treat / Treat with Standard of Care |
| Inflammation Level | CRP < 10 mg/L | 0-90% | | |
| Age | < 60 years | 0-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |
| Combined Episodic + Working Memory Score | Performance beter than normative mean (Zscore conversion = positive score) | 0-90% | Low PND Risk | Do not Treat / Treat with Standard of Care |
| Inflammation Level | CRP < 10 mg/L | 0-90% | | |
| Age | < 60 years | 0-90% | | |
| BMI | < 30.0 | 0-20% | | |
| Education | > 12 years | 0-20% | | |
| Medication Use (current)^{a} | =0 from risk list | 0-20% | | |
| Disease Diagnoses (current or history)^{b} | =0 from risk list | 0-20% | | |

^{a} Medication Use includes use of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication. ^{b} Disease Diagnoses includes a current diagnosis or history of a psychiatric disorder, a neurological disorder, a diabetes, a metabolic syndrome, an autoimmune disease, a heart disease, a vascular disease, a stroke, an infection, an injury, an asthma, or alcohol abuse

Aspects of the present specification disclose the step of determining a PVCI diagnostic score that identifies or confirms a diagnosis of a PVCI for an individual. In some embodiments, a PVCI diagnostic score is based on a standard deviation deficit of an individual’s episodic memory score from a normative mean. In some embodiments, a PVCI diagnostic score is based on a standard deviation deficit of an individual’s episodic memory score from a normative mean in conjunction with a criterion value one or more risk-related factors. In some embodiments, a PVCI diagnostic score is based on a standard deviation deficit of an individual’s working memory score from a normative mean. In some embodiments, a PVCI diagnostic score is based on a standard deviation deficit of an individual’s working memory score from a normative mean in conjunction with a criterion value one or more risk-related factors. In some embodiments, a PVCI diagnostic score is based on a standard deviation deficit of an individual’s combined episodic memory and working memory score from a normative mean. In some embodiments, a PVCI diagnostic score is based on a standard deviation deficit of an individual’s combined episodic memory and working memory score from a normative mean in conjunction with a criterion value one or more risk-related factors.

In some embodiments, an identification or confirmation of a diagnosis of an individual for a PVCI uses a risk stratification matrix that employs pre-defined threshold criteria values to an episodic memory score, a working memory score and each additional risk-related information marker by which an individual’s PVCI diagnostic score is determined and identification or confirmation of a PVCI is assessed. In some embodiments, pre-defined threshold criteria values identifying or confirming an individual has a PVCI is determined by an episodic memory criterion value and an inflammation criterion value. In aspects of these embodiments, an episodic memory criterion value includes a score of greater than or equal to a standard deviation of one below a normative mean (Zscore conversion is equal to or less than -1) and an inflammation criterion value includes the presence of a CRP blood level of 10 mg/L or more. In some embodiments, pre-defined threshold criteria values identifying or confirming an individual has a PVCI is determined by a working memory criterion value and an inflammation criterion value. In aspects of these embodiments, a working memory criterion value includes a score of greater than or equal to a standard deviation of one below a normative mean (Zscore conversion is equal to or less than -1) and an inflammation criterion value includes the presence of a CRP blood level of 10 mg/L or more. In some embodiments, pre-defined threshold criteria values identifying or confirming an individual has a PVCI is determined by a combined episodic memory and working memory criterion value, and an inflammation criterion value. In aspects of these embodiments, a combined episodic memory and working memory criterion value includes a score of greater than or equal to a standard deviation of one below a normative mean (Zscore conversion is equal to or less than -1); and an inflammation criterion value includes the presence of a CRP blood level of 10 mg/L or more.

In some embodiments, pre-defined threshold criteria values identifying or confirming an individual has a PVCI is determined by an episodic memory criterion value and the presence of two or more factors with one factor being a viral infection criterion value and the other factor being an inflammation criterion value. In aspects of these embodiments, an episodic memory criterion value includes a score of greater than or equal to a standard deviation of one below a normative mean (Zscore conversion is equal to or less than -1); a viral infection criterion value being at least one viral infection in the previous 10 years; and an inflammation criterion value includes the presence of a CRP blood level of 10 mg/L or more.

In some embodiments, pre-defined threshold criteria values identifying or confirming an individual has a PVCI is determined by a working memory criterion value and the presence of two or more factors with one factor being a viral infection criterion value and the other factor being an inflammation criterion value. In aspects of these embodiments, a working memory criterion value includes a score of greater than or equal to a standard deviation of one below a normative mean (Zscore conversion is equal to or less than -1); a viral infection criterion value being at least one viral infection in the previous 10 years; and an inflammation criterion value includes the presence of a CRP blood level of 10 mg/L or more.

In some embodiments, pre-defined threshold criteria values identifying or confirming an individual has a PVCI is determined by a combined episodic memory and working memory criterion value, and the presence of two or more factors with one factor being a viral infection criterion value and the other factor being an inflammation criterion value. In aspects of these embodiments, a combined episodic memory and working memory criterion value includes a score of greater than or equal to a standard deviation of one below a normative mean (Zscore conversion is equal to or less than -1); a viral infection criterion value being at least one viral infection in the previous 10 years; and an inflammation criterion value includes the presence of a CRP blood level of 10 mg/L or more.

In some embodiments, pre-defined threshold criteria values identifying or confirming an individual has a PVCI is determined by an episodic memory criterion value and the presence of two or more factors with one factor being a viral infection criterion value and the other factor being the anti-viral medication use criterion value. In aspects of these embodiments, an episodic memory criterion value includes a score of greater than or equal to a standard deviation of one below a normative mean (Zscore conversion is equal to or less than -1); a viral infection criterion value being at least one viral infection in the previous 10 years; and an antiviral medication use criterion value includes the use in the previous 8 weeks of an antiviral medication.

In some embodiments, pre-defined threshold criteria values identifying or confirming an individual has a PVCI is determined by a working memory criterion value and the presence of two or more factors with one factor being a viral infection criterion value and the other factor being the anti-viral medication use criterion value. In aspects of these embodiments, a working memory criterion value includes a score of greater than or equal to a standard deviation of one below a normative mean (Zscore conversion is equal to or less than -1); a viral infection criterion value being at least one viral infection in the previous 10 years; and an antiviral medication use criterion value includes the use in the previous 8 weeks of an antiviral medication.

In some embodiments, pre-defined threshold criteria values identifying or confirming an individual has a PVCI is determined by a combined episodic memory and working memory criterion value, and the presence of two or more factors with one factor being a viral infection criterion value and the other factor being the anti-viral medication use criterion value. In aspects of these embodiments, a combined episodic memory and working memory criterion value includes a score of greater than or equal to a standard deviation of one below a normative mean (Zscore conversion is equal to or less than -1); a viral infection criterion value being at least one viral infection in the previous 10 years; and an antiviral medication use criterion value includes the use in the previous 8 weeks of an antiviral medication.

In some embodiments, pre-defined threshold criteria values identifying or confirming an individual at has a PVCI is determined by an episodic memory criterion value and the presence of three or more factors with one factor being an inflammation criterion value, another factor being a viral infection criterion value, and the third factor being an anti-viral medication use criterion value. In aspects of these embodiments, an episodic memory criterion value includes a score of greater than or equal to a standard deviation of one below a normative mean (Zscore conversion is equal to or less than -1); a viral infection criterion value being at least one viral infection in the previous 10 years; an antiviral medication use criterion value includes the use in the previous 8 weeks of an antiviral medication; and an inflammation criterion value includes the presence of a CRP blood level of 10 mg/L or more.

In some embodiments, pre-defined threshold criteria values identifying or confirming an individual at has a PVCI is determined by a working memory criterion value and the presence of three or more factors with one factor being an inflammation criterion value, another factor being a viral infection criterion value, and the third factor being an anti-viral medication use criterion value. In aspects of these embodiments, a working memory criterion value includes a score of greater than or equal to a standard deviation of one below a normative mean (Zscore conversion is equal to or less than -1); a viral infection criterion value being at least one viral infection in the previous 10 years; an antiviral medication use criterion value includes the use in the previous 8 weeks of an antiviral medication; and an inflammation criterion value includes the presence of a CRP blood level of 10 mg/L or more.

In some embodiments, pre-defined threshold criteria values identifying or confirming an individual at has a PVCI is determined by a combined episodic memory and working memory criterion value, and the presence of three or more factors with one factor being an inflammation criterion value, another factor being a viral infection criterion value, and the third factor being an anti-viral medication use criterion value. In aspects of these embodiments, a combined episodic memory and working memory criterion value includes a score of greater than or equal to a standard deviation of one below a normative mean (Zscore conversion is equal to or less than -1); a viral infection criterion value being at least one viral infection in the previous 10 years; an antiviral medication use criterion value includes the use in the previous 8 weeks of an antiviral medication; and an inflammation criterion value includes the presence of a CRP blood level of 10 mg/L or more.

In addition, the risk stratification matrix can further stratify the PVCI diagnosis-score of an individual to identify or confirm the presence of a PVCI. In some embodiments, a risk stratification matrix disclosed herein can apply a weighted score to each threshold criterion value assigned to an episodic memory score, a working memory score, a combined episodic and working memory score, each additional risk-related factor, or any combination thereof. In some embodiments, the weighted score assigned to each threshold criterion value by the risk stratification matrix is developed from data obtained from information compiled on a PVCI, such as, e.g., information obtained from observational studies, interventional studies, and clinical studies.

If a PVCI diagnosis-score of the individual meets certain predefined threshold criteria values, a risk stratification matrix disclosed herein will indicate that the individual has a PVCI. In addition, depending on which predefined threshold criteria values are meet, a risk stratification matrix disclosed herein can qualify the degree of cognitive impairment.

In some embodiments, a weighted score assigned to each threshold criterion value by the risk stratification matrix for an episodic memory score, a working memory score, a combined episodic and working memory score, and each additional risk-related factor to ascertain a PVCI diagnosis-score that identifies or confirms the presence of a PVCI in an individual is based on Table 2.

TABLE 2

Risk Stratification Matrix for PVCI Diagnosis-Score Ascertainment and PVCI Diagnosis

| Risk Factor | Individual’s Risk-Related Information | Weighted Score | PVCI Diagnostic Score | Therapy Recommendation |
|---|---|---|---|---|
| Episodic Memory Score | ≥ 1SD deficit compared to normative mean (Zscore conversion ≤ -1) | 100% | PVCI Present | Treat with Fenofibrate |
| Working Memory Score | ≥ 1SD deficit compared to normative mean (Zscore conversion ≤ -1) | 100% | PVCI Present | Treat with Fenofibrate |
| Combined Episodic + Working Memory Score | ≥ 1SD deficit compared to normative mean (Zscore conversion ≤ -1) | 100% | PVCI Present | Treat with Fenofibrate |
| Episodic Memory Score | ≥ 1SD deficit compared to normative mean (Zscore conversion ≤ -1) | 60-90% | PVCI Present | Treat with Fenofibrate |
| Inflammation Level | CRP > 10 mg/L | 35-90% | | |
| Viral Infection Diagnoses (current or history) | ≥ 1 infection in previous 10 years | 5-20% | | |
| Working Memory Score | ≥ 1SD deficit compared to normative mean (Zscore conversion ≤ -1) | 60-90% | PVCI Present | Treat with Fenofibrate |
| Inflammation Level | CRP > 10 mg/L | 35-90% | | |
| Viral Infection Diagnoses (current or history) | ≥ 1 infection in previous 10 years | 5-20% | | |
| Combined Episodic + Working Memory Score | ≥ 1SD deficit compared to normative mean (Zscore conversion ≤ -1) | 60-90% | PVCI Present | Treat with Fenofibrate |
| Inflammation Level | CRP > 10 mg/L | 35-90% | | |
| Viral Infection Diagnoses (current or history) | ≥ 1 infection in previous 10 years | 5-20% | | |
| Episodic Memory Score | ≥ 1SD deficit compared to normative mean (Zscore conversion ≤ -1) | 60-90% | PVCI Present | Treat with Fenofibrate |
| Inflammation Level | CRP > 10 mg/L | 30-90% | | |
| Antiviral Medication Use | ≥ 1 in previous 8 weeks | 5-20% | | |
| Viral Infection Diagnoses (current or history) | ≥ 1 infection in previous 10 years | 5-20% | | |
| Working Memory Score | ≥ 1SD deficit compared to normative mean (Zscore conversion ≤ -1) | 60-90% | PVCI Present | Treat with Fenofibrate |
| Inflammation Level | CRP > 10 mg/L | 30-90% | | |
| Antiviral Medication Use | ≥ 1 in previous 8 weeks | 5-20% | | |
| Viral Infection Diagnoses (current or history) | ≥ 1 infection in previous 10 years | 5-20% | | |
| Combined Episodic + Working Memory Score | ≥ 1SD deficit compared to normative mean (Zscore conversion ≤ -1) | 60-90% | PVCI Present | Treat with Fenofibrate |
| Inflammation Level | CRP > 10 mg/L | 30-90% | | |
| Antiviral Medication Use | ≥ 1 in previous 8 weeks | 5-20% | | |
| Viral Infection Diagnoses (current or history) | ≥ 1 infection in previous 10 years | 5-20% | | |
| Episodic Memory Score | < 1SD deficit compared to normative mean (Zscore conversion ≥ -1) | 0-90% | PVCI not Present | No PVCI - Do not Treat |
| Inflammation Level | CRP < 10 mg/L | 0-90% | | |
| Viral Infection Diagnoses (current or history) | < 1 infection in previous 10 years | 0-20% | | |
| Antiviral Medication Use | < 1 in previous 8 weeks | 0-20% | | |
| Working Memory Score | < 1SD deficit compared to normative mean (Zscore conversion ≥ -1) | 0-90% | PVCI not Present | No PVCI - Do not Treat |
| Inflammation Level | CRP < 10 mg/L | 0-90% | | |
| Viral Infection Diagnoses (current or history) | < 1 infection in previous 10 years | 0-20% | | |
| Antiviral Medication Use | < 1 in previous 8 weeks | 0-20% | | |
| Combined Episodic + Working Memory Score | < 1SD deficit compared to normative mean (Zscore conversion ≥ -1) | 0-90% | PVCI not Present | No PVCI - Do not Treat |
| Inflammation Level | CRP < 10 mg/L | 0-90% | | |
| Viral Infection Diagnoses (current or history) | < 1 infection in previous 10 years | 0-20% | | |
| Antiviral Medication Use | < 1 in previous 8 weeks | 0-20% | | |

As noted in Table 1 and Table 2, the disclosed systems, methods, and uses may therefore apply weights, or weighted scores, based on criterion values of the risk stratification matrices. This is performed in association with one or both of an episodic memory score and a working memory score, to comprise a matrix which is evaluated against the episodic memory score and/or working memory score by which to ascertain a PND risk score to determine an at-risk susceptibility of an individual for a PND, and to ascertain a PVCI diagnostic score to determine or confirm the diagnosis of an individual for a PVCI. In the case of PND, criterion such as inflammation level, age, BMI, education, medication use, and disease diagnoses are variables that comprise a clinical or patient-based approach to ascertain a PND risk score. In the case of PVCI, criterion such as inflammation level, antiviral medication use, and viral infection diagnosis or history are variables that comprise a clinical or patient-based approach to ascertain a PVCI diagnostic score. In both cases, these variables are predictors which, when assigned weights or weighted scores based on the criterion values, are indicators whereby the mathematical functions comprising the risk stratification are able to model the risk score for each patient.

It is to be understood that other predictive variables may also be utilized, either instead of or in addition to those listed herein, and therefore the present invention is not to be limited to any particular predictive variable or set of predictive variables referenced herein. For example, other medications and other disease diagnoses may be relevant to the PND risk score or the PVCI diagnostic score and may be modelled in the risk stratification matrix and may themselves be assigned different weighted scores based on particular characteristics thereof. Non-limiting predictive variables include an additional or an alternative cognitive domain, including subdomains of memory, psychomotor speed, attention, executive function, social and emotional cognition, preoperative delirium, a depression scale, an anxiety scale, a pain scale, a sedation scale, a general health scale, type of surgery, length of surgery, type of anaesthesia, length of anaesthesia, a medication scale, a drug scale, one or more neuroimaging parameters, one or more inflammatory cytokines, a quality of life assessment, a disability (ADL/IADL), demographic information, socioeconomic information, one or more comorbidities, one or more lifestyle risks, frailty, hypotension, respiratory complications and IQ. Regardless, the matrix against which episodic memory scores and working memory scores may include other variables and having other associated weighed scores.

A risk stratification matrix is an approach to modeling such variables that is an application of mathematical functions that assess the importance of relevant clinical characteristics for PND and PCVI, such as, e.g., an episodic memory score, a working memory score, a combined episodic memory and working memory score, one or more risk-related factors, or any combination thereof. These mathematical functions are used to determine an associated weighed score for each variable. It is to be understood however that such weighed scores may be adjusted according to different criterion values, and therefore the mathematical functions may include one or more techniques that evaluate the associated weighed scores based on variances within each variable. Other mathematical functions may also be used to evaluate such associated weighed scores, for example functions that account for temporal changes in variables, such as a patient’s BMI or variances in amounts of medication, that may be relevant to the overall assessment of PND or PVCI. Still further, other mathematical approaches may be utilized, such as regression models for estimating relationships between variables, Bayesian models for updating weights or weighted scores as more clinical data becomes available from a wider array of patients, and any other approach which improves the ability to determine the relevancy of variables and the process for weighed their importance to achieve more accurate risk scores, as well as the ability to evaluate episodic and/or working memory scores against a matrix of such variables.

Mathematical functions such as regression and Bayesian analysis are techniques of machine learning in which computing systems can be applied to improve upon the determinations of relevancy of variables, and the process for weighed their importance. Therefore, the present invention may include applications of machine learning and artificial intelligence to assessing risk scores for PND and PVCI. Such applications of machine learning and artificial intelligence may be represented in a machine learning modeling layer in conjunction with the risk stratification matrix.

The machine learning modelling layer may be comprised of one or more neural networks that further evaluate variables to perform the risk stratification matrix and other specific algorithms attendant to ascertaining a PND or PVCI risk score. Neural networks are comprised of, and variables may be assigned to, nodes which are computational units having one or more biased input/output connections, which are transfer (or activation) functions that combine the inputs in some way, and an output connection. The nodes are then organized into layers that form a neural network. There are many types of neural networks, which are computing systems that “learn” to perform tasks without being programmed with task-specific rules, based on examples. Neural networks are based on arrays of connected, aggregated nodes (or “neurons”) that transmit signals to each other in multiple layers over connections. Connections as noted above are activation or transfer functions that “fire” these nodes and combine inputs according to mathematical equations or formulas. Different types of neural networks generally have different configurations of these layers of connected, aggregated nodes, but they can generally be described as an input layer, a middle or ‘hidden’ layer, and an output layer. These layers may perform different transformations on their various inputs, using different mathematical calculations or functions. Signals travel between these layers, from the input layer to the output layer via the middle layer, and may traverse layers, and nodes, multiple times. Signals are transmitted between nodes over connections, and the output of each node is calculated in a non-linear function that sums all of the inputs to that node. Weight matrices and biases are typically applied to each node, and each connection, and these weights and biases are adjusted as the neural network processes inputs and transmits them across the nodes and connections. These weights represent increases or decreases in the strength of a signal at a particular connection. Additionally, nodes may have a threshold, such that a signal is sent only if the aggregated output at that node crosses that threshold. Weights represent how long an activation function takes, while biases represent when, in time, such a function starts; together, they help gradients minimize over time. At least in the case of weights, they can be initialized and change (i.e., decay) over time, as a system learns what weights should be, and how they should be adjusted, for example based on a particular value of a variable. In other words, neural networks evolve as they learn, and the mathematical formulas and functions that comprise neural networks design can change over time as a system improves itself.

Aspects of the present specification disclose the step of providing a treatment recommendation based on the individual’s at-risk susceptibility to a PND. Both preclinical and clinical studies suggest an underlying role of immune activation and neuroinflammation in the etiology of a PND. For example, these studies have repeatedly demonstrated direct correlations between inflammatory response and cognitive function following surgery and people with higher pro-inflammatory cytokines post-surgery are at higher risk for a PND. Mechanistically, surgery itself can be a trigger of a PND as the resulting tissue damage induces inflammatory processes, provoking the release of IL-1 and TNF-α from endothelial cells and phagocytes, triggering a cascade of downstream signalling events. Although the blood brain barrier (BBB) does tightly regulate the transit of inflammatory factors into the CNS, peripheral inflammatory states can compromise the integrity of the BBB. Such compromise enables CNS entry of peripheral inflammatory factors causing neuroinflammation which in turn triggers CNS expression of inflammatory cytokines, and further neuroinflammation in the CNS. The hippocampus contains the largest density of cytokine (inflammatory) receptors and is especially vulnerable to the detrimental effects of inflammation. This brain region is well established for its role in learning and memory formation. Thus, elevated levels of brain inflammation can adversely affect the hippocampus causing diminished function of these cognitive processes.

In addition to neuroinflammation as a consequence of the surgical procedure, the use of anaesthetics pre-surgery is thought to contribute to the onset of a PND as they penetrate the CNS and have direct activity on neuronal signalling and activity. Anaesthetics exhibit time and dose-dependent toxicity to nerves resulting in clinically relevant nerve damage. In addition, anaesthetics perioperative immunosuppression is observed in individuals is attributable to anaesthesia and the neuroendocrine stress exerted through activation of the autonomic nervous system and the hypothalamic-pituitary-adrenal (HPA) axis. Thus, the use of anaesthetics during surgery also appears to cause neuroinflammation.

Although it may accelerate the development of a pre-existing neurodegenerative disease, a PND is not in itself a neurodegenerative disease such as Alzheimer Disease or Parkinson’s Disease. As discussed above a PND appears to be caused via surgical- or anesthetic-induced insult that triggers a neuroinflammatory response in the brain affecting the learning and memory functions of the hippocampus. The cognitive impairment associated with a PND is either transient or chronic. On the other hand, neurodegenerative diseases are not temporary, and the chronic cognitive impairment seen in some PND individuals does not coincide with a process of impairment that is degenerative in nature. Thus, a PND is not a neurodegenerative disease.

Fibrates are a class of amphipathic carboxylic acids with lipid level modifying properties. Non-limiting examples of fibrates include bezafibrate, ciprofibrate, clinofibrate, clofibrate, clofibride, fenofibrate, fenofibric acid, gemfibrozil, ronifibrate, and simfibrate. These drugs are used for a range of metabolic disorders, primarily hypercholesterolemia (high cholesterol), and are therefore considered hypolipidemic agents. Fibrates activate peroxisome proliferator-activated receptors (PPARs), especially PPARα. PPARs are a class of intracellular receptors that modulate carbohydrate and fat metabolism and adipose tissue differentiation.

Besides PPAR agonism, the anti-inflammatory effects of fibrates are influenced non-steroidal anti-inflammatory drugs (NSAIDs) effects due to their ability to inhibit selective cyclooxygenase 2 (COX 2) activity, as well as their cannabinoid receptor type 2 (CB2) agonism. CB2 is expressed in peripheral immune cells such as monocytes, macrophages, B-cells, and T-cells as well microglia in the brain. The combination of these three complimentary pharmacologies is being proposed for the complete resolution of inflammation, devoid of any gastric erosion potential and cardiac liability.

Two examples of a fibrate include fenofibrate and fenofibric acid. Fenofibrate is fibric acid derivative comprising fenofibric acid linked to an isopropyl ester. As such, fenofibrate can be considered a prodrug of fenofibric acid. Fenofibrate has a logP of 5.2 whereas fenofibric acid has a logP of 3.9. The chemical structure of these two compounds is shown below. The in-market compound Tricor is a fenofibrate approved for the treatment of abnormal blood lipid levels with marketing authorization supporting its use in extending life expectancy of cardiac compromised individuals.

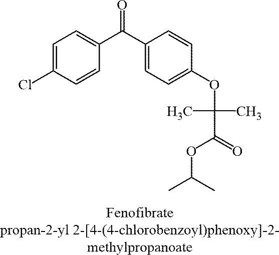

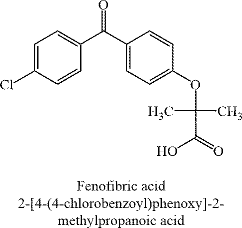

In some embodiments, providing a treatment recommendation based on the individual’s at-risk susceptibility to a PND includes i) no therapy recommendation; or ii) a therapy using a fibrate. In aspects of these embodiments, an at-risk PND assessment indicating that an individual meets the threshold criterion matrix of Table 1, a therapy recommendation includes treating with a fibrate. In other aspects of these embodiments, an at-risk PND assessment indicating that an individual meets the threshold criterion matrix of Table 2, a therapy recommendation includes treating with a fibrate.

In other aspects of these embodiments, an at-risk PND assessment indicating that an individual is classified as having a preoperative neurocognitive disorder, a therapy recommendation includes treating with a fibrate. In yet other aspects of these embodiments, an at-risk PND assessment indicating that an individual is classified as having a delayed postoperative neurocognitive recovery (or early post-operative cognitive dysfunction), a therapy recommendation includes treating with a fibrate. In still other aspects of these embodiments, an at-risk PND assessment indicating that an individual is classified as having a mild postoperative neurocognitive disorder (or long-term post-operative cognitive dysfunction), a therapy recommendation includes treating with a fibrate. In other aspects of these embodiments, an at-risk PND assessment indicating that an individual is classified as having a major postoperative neurocognitive disorder (or long-term post-operative cognitive dysfunction), a therapy recommendation includes treating with a fibrate. In yet other aspects of these embodiments, an at-risk PND assessment indicating that an individual is classified as having a mild neurocognitive disorder (or long-term post-operative cognitive dysfunction), a therapy recommendation includes treating with a fibrate. In still other aspects of these embodiments, an at-risk PND assessment indicating that an individual is classified as having a major neurocognitive disorder (or long-term post-operative cognitive dysfunction), a therapy recommendation includes treating with a fibrate.

Aspects of the present specification disclose a method of treating an individual at risk of or suffering from a PND or a PVCI. In some embodiments, the disclosed method comprises, consists essentially of, or consists of administering to an individual at risk of or suffering from a PND or a PVCI a composition comprising an effective amount of a fibrate. In some embodiments, a composition disclosed herein comprises, consists essentially of, or consists of bezafibrate, ciprofibrate, clinofibrate, clofibrate, clofibride, fenofibrate, fenofibric acid, gemfibrozil, ronifibrate, or simfibrate. In a preferred embodiment, a composition disclosed herein is fenofibrate, or fenofibric acid.

Aspects of the present specification disclose an effective amount of a fibrate for use in treating an individual at risk of or suffering from a PND or a PVCI. In some embodiments, a composition disclosed herein comprises, consists essentially of, or consists of bezafibrate, ciprofibrate, clinofibrate, clofibrate, clofibride, fenofibrate, fenofibric acid, gemfibrozil, ronifibrate, or simfibrate. In a preferred embodiment, a composition disclosed herein is fenofibrate, or fenofibric acid.

Aspects of the present specification disclose use of a fibrate in the manufacture of a medicament for the treatment of an individual at risk of or suffering from a PND or a PVCI. In some embodiments, a composition disclosed herein comprises, consists essentially of, or consists of bezafibrate, ciprofibrate, clinofibrate, clofibrate, clofibride, fenofibrate, fenofibric acid, gemfibrozil, ronifibrate, or simfibrate. In a preferred embodiment, a composition disclosed herein is fenofibrate, or fenofibric acid.

A composition or medicament disclosed herein refers to a composition or medicament comprising an effective concentration of a fibrate disclosed herein. Preferably, a composition or medicament disclosed herein does not produce an adverse, allergic, or other untoward or unwanted reaction when administered to an individual. A composition or medicament disclosed herein is useful for medical and veterinary applications. A composition or medicament disclosed herein can be formulated into any form that enables administration of a composition or medicament disclosed herein in a manner that achieves a desired beneficial effect. In one embodiment, a composition or medicament disclosed herein can be formulated into, e.g., a single-phase formulation, a biphasic formulation comprising a medium phase and a dispersed phase, or a multiphasic formulation. In another embodiment, a composition or medicament disclosed herein can be formulated into, e.g., a liquid composition or medicament, a colloidal composition or medicament, a semi-solid composition or medicament, or a solid composition or medicament. In another embodiment, a composition or medicament disclosed herein can be formulated into, e.g., a liquid aerosol, a foam, an emulsion, a gel, a sol, or a solid sol. A composition or medicament disclosed herein may be formulated as an immediate or a controlled-released formulation. A composition or medicament disclosed herein may be administered to an individual alone, or in combination with other supplementary active compounds, agents, drugs or hormones.

The present specification discloses, in part, an effective amount. With respect to a method or use disclosed herein, a fibrate disclosed herein is used or administered in an effective amount. An effective amount of a fibrate disclosed herein is an amount sufficient to treat a PND or pathology or a PVCI or pathology. In aspects of this embodiment, an effective amount of a fibrate disclosed herein is an amount sufficient to reduce one or more physiological conditions or symptoms associated with a PND or pathology or a PVCI or pathology or an amount sufficient to protect the individual against one or more physiological conditions or symptoms associated with a PND or pathology or a PVCI or pathology. As used herein, the term “effective amount” includes the terms “amount sufficient”, “therapeutically sufficient amount”, “therapeutically effective amount”, “effective dose”, or “therapeutically effective dose” and refers to the minimum amount of a fibrate disclosed herein necessary to achieve the desired therapeutic effect and includes an amount sufficient to reduce or inhibit one or more physiological conditions or symptoms associated with a PND or pathology or a PVCI or pathology.

In aspects of this embodiment, an effective amount of a fibrate disclosed herein reduces or inhibits one or more physiological conditions or symptoms associated with a PND or pathology or a PVCI or pathology by, e.g., at least 10%, at least 20%, at least 30%, at least 40%, at least 50%, at least 60%, at least 70%, at least 80%, at least 90% or at least 100%. In other aspects of this embodiment, an effective amount of a fibrate disclosed herein reduces or inhibits one or more physiological conditions or symptoms associated with a PND or pathology or a PVCI or pathology by, e.g., at most 10%, at most 20%, at most 30%, at most 40%, at most 50%, at most 60%, at most 70%, at most 80%, at most 90% or at most 100%. In yet other aspects of this embodiment, an effective amount of a fibrate disclosed herein reduces or inhibits one or more physiological conditions or symptoms associated with a PND or pathology or a PVCI or pathology by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, an effective amount of a fibrate disclosed herein reduces or inhibits one or more physiological conditions or symptoms associated with a PND or pathology or a PVCI or pathology for, e.g., at least one week, at least one month, at least two months, at least three months, at least four months, at least five months, at least six months, at least seven months, at least eight months, at least nine months, at least ten months, at least eleven months, or at least twelve months.

The actual effective amount of a fibrate disclosed herein to be used or administered to an individual can be determined by a person of ordinary skill in the art by taking into account factors, including, without limitation, the type of PND or pathology or a PVCI or pathology, the particular physiological conditions or symptoms associated with a PND or pathology or a PVCI or pathology, the cause of a PND or pathology or a PVCI or pathology, the severity of a PND or pathology or a PVCI or pathology, the degree of relief desired for a PND or pathology or a PVCI or pathology, the duration of relief desired for a PND or pathology or a PVCI or pathology, the particular fibrate used in a disclosed method or use, the rate of excretion of the particular fibrate used in a disclosed method or use, the pharmacodynamics of the particular fibrate used in a disclosed method or use, the nature of the other compounds to be included in a disclosed method or use, the particular route of administration used in a disclosed method or use, the particular characteristics, history and risk factors of the individual, such as, e.g., age, weight, general health and the like, or any combination thereof. Additionally, where repeated administration of a disclosed method or use is employed, the actual effective amount will further depend upon factors, including, without limitation, the frequency of administration, the half-life of a fibrate disclosed herein used in a disclosed method or use, or any combination thereof.

The actual effective amount of a fibrate disclosed herein is determined by routine screening procedures employed to evaluate an activity and efficacy of a fibrate disclosed herein. Such screening procedures are well known by those skilled in the art. For example, it is known by a person of ordinary skill in the art that an effective amount of a fibrate disclosed herein can be extrapolated from in vitro assays and in vivo administration studies using animal models prior to administration to humans. Wide variations in the necessary effective amount are to be expected in view of the differing efficiencies of the various routes of administration. For instance, oral use or administration generally would be expected to require higher dosage levels than use or administration by intravenous or intravitreal injection. Variations in these dosage levels can be adjusted using standard empirical routines of optimization, which are well-known to a person of ordinary skill in the art. In addition, variations in dosage levels can be adjusted using standard empirical routines of optimization, which are well-known to a person of ordinary skill in the art. It is expected that a fibrate disclosed herein having a higher level of activity and/or efficacy can be used in a smaller effective amount, while a fibrate having a lower level of activity and/or efficacy may require a larger effective amount in order to achieve the same controlling effect. Such effective amounts can be determined by routine assays/measurements of activity and/or efficacy for a fibrate disclosed herein. The precise therapeutically effective dosage levels and patterns are preferably determined by the attending healthcare professional in consideration of the above-identified factors.

Dosing can be single dosage or cumulative (serial dosing), and can be readily determined by one skilled in the art. For instance, treatment of a PND or pathology or a PVCI or pathology may comprise a one-time administration of a fibrate used in a disclosed method or use. As a non-limiting example, a fibrate used in a disclosed method or use can be administered once to an individual, e.g., as a single injection or deposition. Alternatively, treatment of a PND or pathology or a PVCI or pathology may comprise multiple administrations of a fibrate used in a disclosed method or use to carried out over a range of time periods, such as, e.g., daily, every other day, every third of day, once a week, multiple times per week, once a month, multiple times per month, once a year or multiple times per year. As a non-limiting example, a fibrate used in a disclosed method or use can be administered once daily to an individual. The timing of administration can vary from individual to individual, depending upon such factors as the severity of an individual’s symptoms. For example, fibrate used in a disclosed method or use can be administered to an individual daily, every other day, every third of day, once a week, multiple times per week, once a month, multiple times per month, once a year or multiple times per year for an indefinite period of time, or until the individual no longer requires treatment of a PND or pathology or a PVCI or pathology. A person of ordinary skill in the art will recognize that the condition of the individual can be monitored throughout the course of treatment and that use or administration of the fibrate used in a disclosed method or use can be adjusted accordingly.

Aspects of the present specification can also be described by the following embodiments:

1. A method of identifying an individual at risk of a perioperative neurocognitive disorder, the method comprising: a) determining an episodic memory criterion value, a working memory criterion value, or a combined episodic memory and a working memory criterion value for the individual; b) determining a PND risk-score by comparing the episodic memory criterion value, the working memory criterion value, or the combined episodic memory and working memory criterion value to a normative mean; and c) determining an at-risk susceptibility of the individual to perioperative neurocognitive disorder by comparing the PND risk-score to pre-defined threshold criteria. Alternatively, steps b) and c) are carried out using an episodic memory criterion value, a working memory criterion value, or a combined episodic memory and a working memory criterion value that has already been determined for the individual. Hence, a method of identifying an individual at risk of a perioperative neurocognitive disorder can comprise: a) providing an episodic memory criterion value, a working memory criterion value, or a combined episodic memory and a working memory criterion value which has been determined for the individual; b) determining a PND risk-score by comparing the episodic memory criterion value, the working memory criterion value, or the combined episodic memory and working memory criterion value to a normative mean; and c) determining an at-risk susceptibility of the individual to perioperative neurocognitive disorder by comparing the PND risk-score to pre-defined threshold criteria.

2. The method of embodiment 1, wherein the episodic memory criterion value is obtained by measuring the episodic memory of an individual using an episodic memory test.

3. The method of embodiment 1, wherein the working memory criterion value is obtained by measuring the working memory of the individual using a working memory test.

4. The method of embodiment 1, wherein the combined episodic memory and working memory criterion value is obtained by measuring both the episodic memory of the individual using an episodic memory test and the working memory of the individual using a working memory test.

5. The method of embodiment 2 or 4, wherein episodic memory test comprises a list learning test, a recognition memory test, or a paired associate learning test.

6. The method of embodiment 3 or 4, wherein working memory test comprises a spatial working memory test, a spatial span test, a digit sequencing test, or a N-back test.

7. The method of any one of embodiments 1-6 further comprising, prior to step (b), determining a weighed score for the episodic memory criterion value, the working memory criterion value, or the combined episodic memory and working memory criterion value.

8. The method of any one of embodiments 1-7, wherein the at-risk susceptibility of the individual is identified as being at high risk for a PND when: a) the episodic memory criterion value is greater than or equal to a standard deviation of one below a normative mean; or b) the working memory criterion value is greater than or equal to a standard deviation of one below a normative mean; or c) the combined episodic memory and working memory criterion value is greater than or equal to a standard deviation of one below a normative mean.

9. The method of any one of embodiments 1-7, wherein the at-risk susceptibility of the individual is identified as being at medium risk for a PND when: a) the episodic memory criterion value is below the normative mean but less than one standard deviation below the normative mean; or b) the working memory criterion value is below the normative mean but less than one standard deviation below the normative mean; or c) the combined episodic memory and working memory criterion value is below the normative mean but less than one standard deviation below the normative mean.

10. The method of any one of embodiments 1-9 further comprising, prior to step (b), determining a criterion value of one or more risk-related factors for the perioperative neurocognitive disorder in the individual.

11. The method of embodiment 10, wherein determining a PND risk-score is done in conjunction with comparing the criterion value for each of the one or more risk-related factors to a normative mean.

12. The method of embodiment 10 or 11, wherein the one or more risk-related factors comprise an inflammation criterion value, an age criterion value, a body mass index (BMI) criterion value, an education criterion value, a medication use criterion value, a disease diagnosis or history criterion value, or any combination thereof.

13. The method of any one of embodiments 10-12 further comprising determining a weighed score for the criterion value of each of the one or more risk-related factors.

14. The method of any one of embodiments 1-13, wherein the PND risk-score is determined using predetermined threshold criteria of Table 1.

15. The method of any one of embodiments 10-14, wherein the at-risk susceptibility of the individual is identified as being at very high risk for a PND when: a) the episodic memory criterion value is greater than or equal to a standard deviation of one below a normative mean and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse; or b) the working memory criterion value is greater than or equal to a standard deviation of one below a normative mean and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse; or c) the combined episodic memory and working memory criterion value is greater than or equal to a standard deviation of one below a normative mean, and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse.

16. The method of any one of embodiments 10-14, wherein the at-risk susceptibility of the individual is identified as being at high risk for a PND when: a) the episodic memory criterion value is greater than or equal to a standard deviation of one below a normative mean and an inflammation criterion value of a blood level of 10 mg/L or more of CRP; or b) the working memory criterion value is greater than or equal to a standard deviation of one below a normative mean and an inflammation criterion value of a blood level of 10 mg/L or more of CRP; or c) the combined episodic memory and working memory criterion value is greater than or equal to a standard deviation of one below a normative mean and an inflammation criterion value of a blood level of 10 mg/L or more of CRP; or d) the episodic memory criterion value is greater than or equal to a standard deviation of one below a normative mean and an age criterion value of 60 or more years; or e) the working memory criterion value is greater than or equal to a standard deviation of one below a normative mean and an age criterion value of 60 or more years; or f) the combined episodic memory and working memory criterion value is greater than or equal to a standard deviation of one below a normative mean and an age criterion value of 60 or more years.

17. The method of any one of embodiments 10-14, wherein the at-risk susceptibility of the individual is identified as being at medium risk for a PND when: a) the episodic memory criterion value is below the normative mean but less than one standard deviation below the normative mean and an inflammation criterion value of a blood level of 10 mg/L or more of CRP; or b) the working memory criterion value is below the normative mean but less than one standard deviation below the normative mean and an inflammation criterion value of a blood level of 10 mg/L or more of CRP; or c) the combined episodic memory and working memory criterion value that is below the normative mean but less than one standard deviation below the normative mean, and an inflammation criterion value of a blood level of 10 mg/L or more of CRP; or d) the episodic memory criterion value is below the normative mean but less than one standard deviation below the normative mean and an age criterion value of 60 or more years; or e) the working memory criterion value is below the normative mean but less than one standard deviation below the normative mean and an age criterion value of 60 or more years; or f) the combined episodic memory and working memory criterion value is below the normative mean but less than one standard deviation below the normative mean, and an age criterion value of 60 or more years.

18. The method of any one of embodiments 10-14, wherein the at-risk susceptibility of the individual is identified as being at low risk for a PND when: a) the episodic memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history; or b) the working memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history; or c) the combined episodic memory and working memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history.

19. The method of any one of embodiments 1-18, wherein at-risk susceptibility of the individual classifies the individual as recommending additional monitoring, recommending treatment for the PND before surgery, and/or recommending treatment for the PND after surgery.

20. The method of any one of embodiments 1-19, wherein at-risk severity classifies the individual as having a preoperative neurocognitive disorder, a postoperative delirium, a delayed postoperative neurocognitive recovery, a mild postoperative neurocognitive disorder, a major postoperative neurocognitive disorder, a mild neurocognitive disorder, or a major neurocognitive disorder.

21. The method of any one of embodiments 1-20, further provides a treatment recommendation based on the at-risk susceptibility of the individual.

22. The method of embodiment 21, wherein the treatment recommendation includes a no therapy recommendation or a therapy using a fibrate.

23. The method of embodiment 21, wherein when the at-risk susceptibility of the individual is identified as being at very high risk for a PND, at high risk for a PND, or at medium risk for a PND, a therapy recommendation includes treatment with one or more fibrates.

24. The method of embodiment 19, wherein a) when the at-risk susceptibility of the individual indicates that the individual has a preoperative neurocognitive disorder, a therapy recommendation includes treatment with one or more fibrates; or b) when the at-risk susceptibility of the individual indicates that the individual has a delayed postoperative neurocognitive recovery, a therapy recommendation includes treatment with one or more fibrates; or c) when the at-risk susceptibility of the individual indicates that the individual has a mild postoperative neurocognitive disorder, a therapy recommendation includes treatment with one or more fibrates; or d) when the at-risk susceptibility of the individual indicates that the individual has a major postoperative neurocognitive disorder, a therapy recommendation includes treatment with one or more fibrates; or e) when the at-risk susceptibility of the individual indicates that the individual has a mild neurocognitive disorder, a therapy recommendation includes treatment with one or more fibrates; or f) when the at-risk susceptibility of the individual indicates that the individual has a major neurocognitive disorder, a therapy recommendation includes treatment with one or more fibrates.

25. The method of embodiment 19, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

26. A method of identifying and/or confirming an individual at risk of a post-viral cognitive impairment (PVCI), the method comprising: a) determining an episodic memory criterion value, a working memory criterion value, or a combined episodic memory and a working memory criterion value for the individual; b) determining a PVCI diagnostic-score by comparing the episodic memory score, the working memory score, or the combined episodic memory and a working memory criterion value to a normative mean; and c) identifying or confirming a diagnosis in the individual of the PVCI by comparing the PVCI diagnostic-score to pre-defined threshold criteria. Alternatively, steps b) and c) are carried out using an episodic memory criterion value, a working memory criterion value, or a combined episodic memory and a working memory criterion value that has already been determined for the individual. Hence, a method of identifying and/or confirming an individual at risk of a post-viral cognitive impairment (PVCI) can comprise: a) providing an episodic memory criterion value, a working memory criterion value, or a combined episodic memory and a working memory criterion value which has been determined for the individual; b) determining a PVCI diagnostic-score by comparing the episodic memory score, the working memory score, or the combined episodic memory and a working memory criterion value to a normative mean; and c) identifying or confirming a diagnosis in the individual of the PVCI by comparing the PVCI diagnostic-score to pre-defined threshold criteria.

27. The method of embodiment 26, wherein the episodic memory criterion value is obtained by measuring the episodic memory of an individual using an episodic memory test.

28. The method of embodiment 26, wherein the working memory criterion value is obtained by measuring the working memory of the individual using a working memory test.

29. The method of embodiment 26, wherein the combined episodic memory and working memory criterion value is obtained by measuring both the episodic memory of the individual using an episodic memory test and the working memory of the individual using a working memory test.

30. The method of embodiment 27 or 29, wherein episodic memory test comprises a list learning test, a recognition memory test, or a paired associate learning test.

31. The method of embodiment 28 or 29, wherein working memory test comprises a spatial working memory test, a spatial span test, a digit sequencing test, or a N-back test.

32. The method of any one of embodiments 26-31 further comprising, prior to step (b), determining a weighed score for the episodic memory criterion value, the working memory criterion value, or the combined episodic memory and working memory criterion value.

33. The method of any one of embodiments 26-32, wherein the PVCI diagnosis is identified or confirmed when: a) the episodic memory criterion value is greater than or equal to a standard deviation of one below a normative mean; or b) the working memory criterion value is greater than or equal to a standard deviation of one below a normative mean; or c) the combined episodic memory and working memory criterion value is greater than or equal to a standard deviation of one below a normative mean.

34. The method of any one of embodiments 26-33 further comprising, prior to step (b), determining a criterion value of one or more risk-related factors for the PVCI in the individual.

35. The method of embodiment 34, wherein determining the PVCI diagnostic-score is done in conjunction with comparing the criterion value for each of the one or more risk-related factors to a normative mean.

36. The method of embodiment 34 or 35, wherein the one or more risk-related factors comprise an inflammation criterion value, an anti-viral medication use criterion value, a viral infection diagnosis or history criterion value, or any combination thereof.

37. The method of any one of embodiments 34-36 further comprising determining a weighed score for the criterion value of each of the one or more risk-related factors.

38. The method of any one of embodiment 26-37, wherein the wherein the PVCI diagnostic-score is determined using predetermined threshold criteria of Table 2.

39. The method of any one of embodiment 20-27, wherein the PVCI diagnosis is identified or confirmed when: a) the episodic memory criterion value is greater than or equal to a standard deviation of one below a normative mean and there are one or more at-risk factors present; or b) the working memory criterion value is greater than or equal to a standard deviation of one below a normative mean and there are one or more at-risk factors present; or c) the combined episodic memory and working memory criterion value is greater than or equal to a standard deviation of one below a normative mean and there are one or more at-risk factors present; or d) the episodic memory criterion value is below the normative mean but less than one standard deviation below the normative mean and there are one or more at-risk factors present; or e) the working memory criterion value is below the normative mean but less than one standard deviation below the normative mean and there are one or more at-risk factors present; or f) the combined episodic memory and working memory criterion value is below the normative mean but less than one standard deviation below the normative mean and there are one or more at-risk factors present.

40. The method of any one of embodiments 26-39, wherein the PVCI diagnostic score classifies the individual as recommending additional monitoring, and/or recommending treatment for the PVCI after infection.

41. The method of any one of embodiments 26-40, further provides a treatment recommendation based on the at-risk susceptibility of the individual.

42. The method of embodiment 41, wherein the treatment recommendation includes a no therapy recommendation or a therapy using a fibrate.

43. The method of embodiment 42, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

Aspects of the present specification can also be described by the following embodiments:

1. A method of identifying an individual at risk of a perioperative neurocognitive disorder, the method comprising: a) determining an episodic memory criterion value, a working memory criterion value, or a combined episodic memory and a working memory criterion value for the individual; b) determining the criterion value of one or more risk-related factors for the perioperative neurocognitive disorder in the individual; c) determining a PND risk-score by i) comparing the episodic memory criterion value, the working memory criterion value, or the combined episodic memory and working memory criterion value to a normative mean; and ii) comparing the criterion value for each of the one or more risk-related factors to a normative mean; and d) determining an at-risk susceptibility of the individual to perioperative neurocognitive disorder by comparing the PND risk-score to pre-defined threshold criteria. Alternatively, steps b), c) and d) are carried out using an episodic memory criterion value, a working memory criterion value, or a combined episodic memory and a working memory criterion value that has already been determined for the individual. Hence, a method of identifying an individual at risk of a perioperative neurocognitive disorder can comprise: a) providing an episodic memory criterion value, a working memory criterion value, or a combined episodic memory and a working memory criterion value which has been determined for the individual; b) determining the criterion value of one or more risk-related factors for the perioperative neurocognitive disorder in the individual; c) determining a PND risk-score by i) comparing the episodic memory criterion value, the working memory criterion value, or the combined episodic memory and working memory criterion value to a normative mean; and ii) comparing the criterion value for each of the one or more risk-related factors to a normative mean; and d) determining an at-risk susceptibility of the individual to perioperative neurocognitive disorder by comparing the PND risk-score to pre-defined threshold criteria.

2. The method of embodiment 1, wherein the episodic memory criterion value is obtained by measuring the episodic memory of an individual using an episodic memory test.

3. The method of embodiment 1, wherein the working memory criterion value is obtained by measuring the working memory of the individual using a working memory test.

4. The method of embodiment 1, wherein the combined episodic memory and working memory criterion value is obtained by measuring both the episodic memory of the individual using an episodic memory test and the working memory of the individual using a working memory test.

5. The method of embodiment 2 or 4, wherein episodic memory test comprises a list learning test, a recognition memory test, or a paired associate learning test.

6. The method of embodiment 3 or 4, wherein working memory test comprises a spatial working memory test, a spatial span test, a digit sequencing test, or a N-back test.

7. The method of any one of embodiments 1-6, wherein the one or more risk-related factors comprise an inflammation criterion value, an age criterion value, a body mass index (BMI) criterion value, an education criterion value, a medication use criterion value, a disease diagnosis or history criterion value, or any combination thereof.

8. The method of ay one of embodiments 1-7 further comprising determining a weighed score for the episodic memory criterion value, the working memory criterion value, the combined episodic memory and working memory criterion value, the criterion value of each of the one or more risk-related factors, or any combination thereof.

9. The method of any one of embodiments 1-8, wherein the PND risk-score is determined using predetermined threshold criteria of Table 1.

10. The method of any one of embodiments 1-9, wherein the at-risk susceptibility of the individual is identified as being at very high risk for a PND when: a) the episodic memory criterion value is greater than or equal to a standard deviation of one below a normative mean and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse; or b) the working memory criterion value is greater than or equal to a standard deviation of one below a normative mean and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse; or c) the combined episodic memory and working memory criterion value is greater than or equal to a standard deviation of one below a normative mean, and the criterion value of one or more risk-related factors includes an inflammation criterion value indicative of high inflammation, an age criterion value of 60 or more years, a BMI criterion value of 30 or more, an education criterion value of 12 years of formal education or less, an medication use criterion value of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, an anti-Parkinson agent, or an anticholinergic-based medication, and a disease diagnosis or history criterion value of the presence of a psychiatric disorder, a neurological disorder, a diabetes, a metabolic syndrome, an autoimmune disease, a heart disease, a vascular disease, a stroke, an infection, an injury, an asthma, or alcohol abuse.

11. The method of any one of embodiments 1-9, wherein the at-risk susceptibility of the individual is identified as being at high risk for a PND when: a) the episodic memory criterion value is greater than or equal to a standard deviation of one below a normative mean; or b) the working memory criterion value is greater than or equal to a standard deviation of one below a normative mean; or c) the combined episodic memory and working memory criterion value is greater than or equal to a standard deviation of one below a normative mean; or d) the episodic memory criterion value is greater than or equal to a standard deviation of one below a normative mean and an inflammation criterion value of a blood level of 10 mg/L or more of CRP; or e) the working memory criterion value is greater than or equal to a standard deviation of one below a normative mean and an inflammation criterion value of a blood level of 10 mg/L or more of CRP; or f) the combined episodic memory and working memory criterion value is greater than or equal to a standard deviation of one below a normative mean and an inflammation criterion value of a blood level of 10 mg/L or more of CRP; or g) the episodic memory criterion value is greater than or equal to a standard deviation of one below a normative mean and an age criterion value of 60 or more years; or h) the working memory criterion value is greater than or equal to a standard deviation of one below a normative mean and an age criterion value of 60 or more years; or i) the combined episodic memory and working memory criterion value is greater than or equal to a standard deviation of one below a normative mean and an age criterion value of 60 or more years.

12. The method of any one of embodiments 1-9, wherein the at-risk susceptibility of the individual is identified as being at medium risk for a PND when: a) the episodic memory criterion value is below the normative mean but less than one standard deviation below the normative mean; or b) the working memory criterion value is below the normative mean but less than one standard deviation below the normative mean; or c) the combined episodic memory and working memory criterion value is below the normative mean but less than one standard deviation below the normative mean; or d) the episodic memory criterion value is below the normative mean but less than one standard deviation below the normative mean and an inflammation criterion value of a blood level of 10 mg/L or more of CRP; or e) the working memory criterion value is below the normative mean but less than one standard deviation below the normative mean and an inflammation criterion value of a blood level of 10 mg/L or more of CRP; or f) the combined episodic memory and working memory criterion value that is below the normative mean but less than one standard deviation below the normative mean, and an inflammation criterion value of a blood level of 10 mg/L or more of CRP; or g) the episodic memory criterion value is below the normative mean but less than one standard deviation below the normative mean and an age criterion value of 60 or more years; or h) the working memory criterion value is below the normative mean but less than one standard deviation below the normative mean and an age criterion value of 60 or more years; or i) the combined episodic memory and working memory criterion value is below the normative mean but less than one standard deviation below the normative mean, and an age criterion value of 60 or more years.

13. The method of any one of embodiments 1-9, wherein the at-risk susceptibility of the individual is identified as being at low risk for a PND when: a) the episodic memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history; or b) the working memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history; or c) the combined episodic memory and working memory criterion value is above the normative mean and the criterion value of one or more risk-related factors includes an inflammation criterion value of a blood level of less than 10 mg/L of CRP, an age criterion value of less than 60 years old, a BMI criterion value of less than 30, an education criterion value of more than 12 years of formal education, an medication use criterion value of no at-risk medication use, and a disease diagnosis or history criterion value of no at-risk disease diagnosis or history.

14. The method of any one of embodiments 1-13, wherein at-risk susceptibility of the individual classifies the individual as recommending additional monitoring, recommending treatment for the PND before surgery, and/or recommending treatment for the PND after surgery.

15. The method of any one of embodiments 1-14, wherein at-risk severity classifies the individual as having a preoperative neurocognitive disorder, a postoperative delirium, a delayed postoperative neurocognitive recovery, a mild postoperative neurocognitive disorder, a major postoperative neurocognitive disorder, a mild neurocognitive disorder, or a major neurocognitive disorder.

16. The method of any one of embodiments 1-15, further provides a treatment recommendation based on the at-risk susceptibility of the individual.

17. The method of embodiment 16, wherein the treatment recommendation includes a no therapy recommendation or a therapy using a one or more fibrates.

18. The method of embodiment 16, wherein when the at-risk susceptibility of the individual is identified as being at very high risk for a PND, at high risk for a PND, or at medium risk for a PND, a therapy recommendation includes treatment with one or more fibrates.

19. The method of embodiment 15, wherein a) when the at-risk susceptibility of the individual indicates that the individual has a preoperative neurocognitive disorder, a therapy recommendation includes treatment with one or more fibrates; or b) when the at-risk susceptibility of the individual indicates that the individual has a delayed postoperative neurocognitive recovery, a therapy recommendation includes treatment with one or more fibrates; or c) when the at-risk susceptibility of the individual indicates that the individual has a mild postoperative neurocognitive disorder, a therapy recommendation includes treatment with one or more fibrates; or d) when the at-risk susceptibility of the individual indicates that the individual has a major postoperative neurocognitive disorder, a therapy recommendation includes treatment with one or more fibrates; or e) when the at-risk susceptibility of the individual indicates that the individual has a mild neurocognitive disorder, a therapy recommendation includes treatment with one or more fibrates; or f) when the at-risk susceptibility of the individual indicates that the individual has a major neurocognitive disorder, a therapy recommendation includes treatment with one or more fibrates.

20. The method of any one of embodiments 17-19, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

21. A method of identifying and/or confirming an individual at risk of a post-viral cognitive impairment (PVCI), the method comprising: a) determining an episodic memory criterion value, a working memory criterion value, or a combined episodic memory and a working memory criterion value for the individual; b) determining the criterion value of one or more risk-related factors for the post-viral cognitive impairment in the individual; c) determining a PVCI diagnostic-score by i) comparing the episodic memory score, the working memory score, or the combined episodic memory and a working memory criterion value to a normative mean and ii) comparing the criterion value for each of the one or more risk-related factors to a normative mean; and d) identifying or confirming a diagnosis in the individual of the PVCI by comparing the PVCI diagnostic-score to pre-defined threshold criteria. Alternatively, steps b), c) and d) are carried out using an episodic memory criterion value, a working memory criterion value, or a combined episodic memory and a working memory criterion value that has already been determined for the individual. Hence, a method of identifying an individual and/or confirming an individual at risk of a post-viral cognitive impairment (PVCI) can comprise: a) providing an episodic memory criterion value, a working memory criterion value, or a combined episodic memory and a working memory criterion value which has been determined for the individual; b) determining the criterion value of one or more risk-related factors for the post-viral cognitive impairment in the individual; c) determining a PVCI diagnostic-score by i) comparing the episodic memory score, the working memory score, or the combined episodic memory and a working memory criterion value to a normative mean and ii) comparing the criterion value for each of the one or more risk-related factors to a normative mean; and d) identifying or confirming a diagnosis in the individual of the PVCI by comparing the PVCI diagnostic-score to pre-defined threshold criteria.

22. The method of embodiment 21, wherein the episodic memory criterion value is obtained by measuring the episodic memory of an individual using an episodic memory test.

23. The method of embodiment 21, wherein the working memory criterion value is obtained by measuring the working memory of the individual using a working memory test.

24. The method of embodiment 21, wherein the combined episodic memory and working memory criterion value is obtained by measuring both the episodic memory of the individual using an episodic memory test and the working memory of the individual using a working memory test.

25. The method of embodiment 22 or 24, wherein episodic memory test comprises a list learning test, a recognition memory test, or a paired associate learning test.

26. The method of embodiment 23 or 24, wherein working memory test comprises a spatial working memory test, a spatial span test, a digit sequencing test, or a N-back test.

27. The method of any one of embodiments 21-26, wherein the one or more risk-related factors comprise an inflammation criterion value, an anti-viral medication use criterion value, a viral infection diagnosis or history criterion value, or any combination thereof.

28. The method of any one of embodiments 26-31 further comprising, prior to step (b), determining a weighed score for the episodic memory criterion value, the working memory criterion value, the combined episodic memory and working memory criterion value, the criterion value of each of the one or more risk-related factors, or any combination thereof.

29. The method of any one of embodiment 21-28, wherein the wherein the PVCI diagnostic-score is determined using predetermined threshold criteria of Table 2.

30. The method of any one of embodiment 20-27, wherein the PVCI diagnosis is identified or confirmed when: a) the episodic memory criterion value is greater than or equal to a standard deviation of one below a normative mean; or b) the working memory criterion value is greater than or equal to a standard deviation of one below a normative mean; or c) the combined episodic memory and working memory criterion value is greater than or equal to a standard deviation of one below a normative mean; or d) the episodic memory criterion value is greater than or equal to a standard deviation of one below a normative mean and there are one or more at-risk factors present; or e) the working memory criterion value is greater than or equal to a standard deviation of one below a normative mean and there are one or more at-risk factors present; or f) the combined episodic memory and working memory criterion value is greater than or equal to a standard deviation of one below a normative mean and there are one or more at-risk factors present; or g) the episodic memory criterion value is below the normative mean but less than one standard deviation below the normative mean and there are one or more at-risk factors present; or h) the working memory criterion value is below the normative mean but less than one standard deviation below the normative mean and there are one or more at-risk factors present; or i) the combined episodic memory and working memory criterion value is below the normative mean but less than one standard deviation below the normative mean and there are one or more at-risk factors present.

31. The method of any one of embodiments 21-30, wherein the PVCI diagnostic score classifies the individual as recommending additional monitoring, and/or recommending treatment for the PVCI after infection.

32. The method of any one of embodiments 21-31, further provides a treatment recommendation based on the at-risk susceptibility of the individual.

33. The method of embodiment 32, wherein the treatment recommendation includes a no therapy recommendation or a therapy using one or more fibrates.

34. The method of embodiment 33, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

Aspects of the present specification can also be described by the following embodiments:

1. A method of treating a perioperative neurocognitive disorder or a post-viral cognitive impairment by administering a composition comprising one or more fibrates.

2. The method of embodiment 1, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

3. The method of embodiment 1 or 2, wherein the perioperative neurocognitive disorder is a delayed postoperative neurocognitive recovery (or early post-operative cognitive dysfunction), a mild postoperative neurocognitive disorder (or long-term post-operative cognitive dysfunction), a major postoperative neurocognitive disorder (or long-term post-operative cognitive dysfunction), a mild neurocognitive disorder (or long-term post-operative cognitive dysfunction), or a major neurocognitive disorder (or long-term post-operative cognitive dysfunction).

4. A composition comprising one or more fibrates for use in treating a perioperative neurocognitive disorder or a post-viral cognitive impairment.

5. The composition of embodiment 4, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

6. The composition of embodiment 4 or 5, wherein the perioperative neurocognitive disorder is a delayed postoperative neurocognitive recovery (or early post-operative cognitive dysfunction), a mild postoperative neurocognitive disorder (or long-term post-operative cognitive dysfunction), a major postoperative neurocognitive disorder (or long-term post-operative cognitive dysfunction), a mild neurocognitive disorder (or long-term post-operative cognitive dysfunction), or a major neurocognitive disorder (or long-term post-operative cognitive dysfunction).

7. Use of one or more fibrates in the manufacture of a medicament for the treatment of a perioperative neurocognitive disorder or a post-viral cognitive impairment.

8. The use of embodiment 7, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

9. The use of embodiment 7 or 8, wherein the perioperative neurocognitive disorder is a delayed postoperative neurocognitive recovery (or early post-operative cognitive dysfunction), a mild postoperative neurocognitive disorder (or long-term post-operative cognitive dysfunction), a major postoperative neurocognitive disorder (or long-term post-operative cognitive dysfunction), a mild neurocognitive disorder (or long-term post-operative cognitive dysfunction), or a major neurocognitive disorder (or long-term post-operative cognitive dysfunction).

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1 Clinical Study of Risk Predictors of POCD in Geriatric Cohort After Elective Surgery

Secondary analysis was carried out on a dataset collected from a prospective multicentre observational study conducted in Europe. Analysis was carried out in 933 patients who underwent elective surgery with an expected surgical duration greater than or equal to 60 minutes. All patients were at least 65 years old with a Mini-Mental State Examination (MMSE) score of >23. Cognitive evaluations were collected prior to surgery (baseline), at discharge (7-days post-surgery), and at 3-month post-surgery follow-up. Cognitive evaluations include tests from the Cambridge Neuropsychological Test Automated Battery (CANTAB), including Reaction Time (RTI; measuring simple attention), Paired Associates Learning (PAL; measuring episodic memory), Spatial Span (SSP; measuring working memory) and Verbal Recognition Memory (VRM; measuring recognition memory). Each of these cognitive tests produce multiple different outcome measures (e.g., errors made, latency, span length) to allow for the statistical evaluation of the participants performance on each of the tasks.

To determine pre-surgical cognitive impairment (i.e., to determine whether having cognitive impairment before surgery, increases a person’s risk susceptibility for PND), a combined (i.e., composite) risk score was calculated from an episodic memory criterion value and a working memory criterion value using the pre-surgical performance of the PAL task and SSP task, respectively. The outcome measures from each task that were used to generate this combined risk score were: Total Errors Adjusted 8 Shapes for the PAL test (PALTEA8), and Total Errors made for the SSP test (SSPTE). To determine whether each individual participant had pre-surgical cognitive impairment which may put them at risk of PND, the pre-surgical (baseline) data for these two outcome measures (Total Errors Adjusted 8 Shapes for the PAL test, and Total Errors made for the SSP test), were pooled across both the surgical group and control group participants, to serve as a normative data comparison against which each individual participant could be compared. The calculation for this combined episodic memory and working memory criterion value was as follows: for each participant, a Z score for each episodic memory and working memory were first created: Z_{PALTEA8} = - (PALTEA8_{individual} _{participant} _{score}- PALTEA8ₙₒᵣₘₐₜᵢᵥₑ ₘₑₐₙ) / PALTEA8ₙₒᵣₘₐₜᵢᵥₑ _{SD}, and Z_{SSPTE} = -(SSPTE_{individual} participant _{score} - SSPTEₙₒᵣₘₐₜᵢᵥₑ ₘₑₐₙ) / SSPTEₙₒᵣₘₐₜᵢᵥₑ _{SD}. These scores were then calculated together to create the combined risk score relating to memory function: Combined risk score = (Z_{PALTEA8} + Z_{SSPTE})/2.Here, a negative risk score would mean an individual participants performance is worse than the normative mean.

Overall pre-surgical cognitive impairment for each participant was classified as cognitive test performance (on Total Errors Adjusted 8 Shapes for the PAL test, and Total Errors made for the SSP test, combined) that was below (i.e., poorer than) the mean of the normative comparison. Mild-to-moderate pre-surgical impairment was classified as cognitive test performance that was >1 SD below (i.e., more than 1SD worse than) the mean of the normative comparison.

To evaluate whether pre-surgical cognitive impairment (either overall pre-surgical impairment or mild-to-moderate pre-surgical impairment) put at individual at risk for PND, post-surgical changes in cognitive performance were statistically evaluated. To evaluate post-surgical changes in cognitive performance, ultimately to determine whether a participant had a PND or not, a reliable change index (RCI) score was calculated using one outcome measure from each of the CANTAB tasks that were used in the European study. For the RTI task, the correct latency outcome measure was used. For the PAL task, the overall total errors adjusted outcome measure was used. For the SSP task, the span length outcome measure was used. For the VRM task, the immediate free recall of words outcome measure was used. For each of the described cognitive outcome measures, the corresponding RCI was calculated. RCI= (ΔX-ΔXc)/SD(ΔXc) where ΔX refers to the difference in test scores after surgery compared to pre-surgery (baseline) and ΔXc refers to the mean test score difference between the corresponding measurement time points in the non-surgical control group (n= 114) to correct for learning effects and variability in repeated cognitive testing. RCI was then normalised to the standard deviation (SD) of mean differences in the control group SD(ΔXc). An overall RCI score for each participant was defined as the sum of all RCI scores for each cognitive outcome measure, Σ(RCI), in relation to the standard deviation of the sum of RCI scores in the control group, SD(Σ(RCIc) using the following formulation: Composite RCI:Σ(RCI) /SD(Σ(RCIc)). An individual was defined as having a PND if 1) their total RCI score was a negative value (indicating cognitive performance had declined after the surgery had taken place), OR 2) if their total RCI score represented a >0.5SD decline (indicating cognitive performance had declined by at least half a SD after the surgery had taken place).

To generate an overview of cognitive performance on the CANTAB tasks at both pre-surgical (baseline) and post-surgical (3-month follow-up) timepoints, summary descriptive statistics are described in Table 3 for each of the test outcome measure variables, for both surgical and control participants.

TABLE 3

Descriptive statistics for CANTAB outcome measures by time point (pre-surgical baseline, 3 months post-surgery) and participant group (surgical and control)

| CANTAB Cognitive Outcome Measure | Group | Time | N | MEAN | MEDIAN | SD | MIN | MAX |
|---|---|---|---|---|---|---|---|---|
| Reaction Time (outcome measure: correct latency) | Surgical | Baseline | 917 | 331.63 | 301.14 | 117.36 | 0.00 | 1423.94 |
| | | 3 months | 639 | 327.12 | 304.30 | 103.42 | 0.00 | 1023.83 |
| | Control | Baseline | 112 | 319.13 | 295.30 | 142.89 | 0.00 | 1037.26 |
| | | 3 months | 102 | 320.32 | 298.56 | 95.40 | 213.90 | 820.30 |
| Verbal Recognition Memory (outcome measure: free recall) | Surgical | Baseline | 908 | 5.80 | 6.00 | 1.97 | 0.00 | 13.00 |
| | | 3 months | 640 | 6.02 | 6.00 | 1.90 | 0.00 | 14.00 |
| | Control | Baseline | 114 | 6.07 | 6.00 | 1.81 | 2.00 | 12.00 |
| | | 3 months | 102 | 6.15 | 6.00 | 1.62 | 3.00 | 10.00 |
| Verbal Recognition Memory (outcome measure: immediate recognition) | Surgical | Baseline | 908 | 22.63 | 23.00 | 3.64 | 0.00 | 36.00 |
| | | 3 months | 640 | 22.55 | 23.00 | 2.61 | 0.00 | 36.00 |
| | Control | Baseline | 112 | 22.35 | 23.00 | 2.12 | 10.00 | 24.00 |
| | | 3 months | 102 | 22.83 | 23.00 | 1.51 | 16.00 | 24.00 |
| Spatial span (outcome measure: span length) | Surgical | Baseline | 919 | 7.74 | 5.00 | 1.07 | 0.00 | 8.00 |
| | | 3 months | 639 | 4.90 | 5.00 | 1.01 | 0.00 | 8.00 |
| | Control | Baseline | 114 | 5.07 | 5.00 | 1.15 | 0.00 | 8.00 |
| | | 3 months | 102 | 5.20 | 5.00 | 1.06 | 3.00 | 8.00 |
| Spatial span (outcome measure: total errors) | Surgical | Baseline | 920 | 12.19 | 11.00 | 4.97 | 0.00 | 38.00 |
| | | 3 months | 639 | 11.72 | 11.00 | 4.70 | 0.00 | 40.00 |
| | Control | Baseline | 114 | 13.24 | 12.00 | 5.16 | 4.00 | 28.00 |
| | | 3 months | 102 | 12.32 | 12.50 | 4.90 | 4.00 | 38.00 |
| Paired Associates Learning (outcome measure: total errors adjusted) | Surgical | Baseline | 913 | 82.47 | 81.00 | 49.80 | 0.00 | 218.00 |
| | | 3 months | 634 | 73.53 | 74.00 | 48.55 | 1.00 | 218.00 |
| | Control | Baseline | 114 | 59.16 | 63.00 | 46.10 | 2.00 | 190.00 |
| | | 3 months | 102 | 55.26 | 52.50 | 45.02 | 1.00 | 171.00 |
| Paired Associates Learning (outcome measure: total errors adjusted 8 shapes) | Surgical | Baseline | 915 | 50.66 | 70.00 | 27.07 | 0.00 | 71.00 |
| | | 3 months | 634 | 48.27 | 68.00 | 28.17 | 0.00 | 71.00 |
| | Control | Baseline | 114 | 39.48 | 56.00 | 29.46 | 0.00 | 71.00 |
| | | 3 months | 102 | 37.96 | 44.50 | 28.71 | 0.00 | 70.00 |

To generate an overview of cognitive performance on the pre-surgical combined ‘risk’ score of episodic memory and working memory for each of the different classifications of PND (negative RCI score and RCIscore >0.5SD), summary descriptive statistics are provided in Table 4 below. In addition to the combined score, the descriptive statistics are also provided for both episodic memory and working memory independently.

TABLE 4

Descriptive Statistics for CANTAB ‘risk’ score variables, for each PND classification

| Test | Classification | N | MEAN | SD | MIN | MAX |
|---|---|---|---|---|---|---|
| PND = Negative RCI Score | | | | | | |
| Paired Associates Learning: Episodic memory (outcome measure: total errors adjusted 8 shapes) | PND | 289 | 53.83 | 25.53 | 0.00 | 71.00 |
| | No PND | 304 | 49.49 | 27.95 | 0.00 | 71.00 |
| Spatial Span: Working memory (outcome measure: total errors) | PND | 295 | 12.79 | 5.04 | 3.00 | 30.00 |
| | No PND | 308 | 11.55 | 4.75 | 4.00 | 36.00 |
| Combined episodic + working memory score | PND | 289 | -0.1235 | 0.6972 | -2.15 | 1.55 |
| | No PND | 304 | 0.0854 | 0.6701 | -1.85 | 1.55 |
| RCI score (3-month) | PND | 295 | -0.8648 | 0.7232 | -3.83 | -0.004 |
| | No PND | 308 | 0.9099 | 0.7086 | -0.00 | 3.67 |

PND = RCI score >0.5 SD

| Paired Associates Learning: Episodic memory (outcome measure: total errors adjusted 8 shapes) | PND | 179 | 50.67 | 27.31 | 0.00 | 70.00 |
|---|---|---|---|---|---|---|
| | No PND | 414 | 52.10 | 26.69 | 0.00 | 71.00 |
| Spatial Span: Working memory (outcome measure: total errors) | PND | 183 | 13.35 | 5.09 | 4.00 | 30.00 |
| | No PND | 720 | 11.64 | 4.77 | 3.00 | 36.00 |
| Combined episodic + working memory risk score | PND | 179 | -0.1245 | 0.7167 | -2.15 | 1.55 |
| | No PND | 414 | 0.0303 | 0.6749 | -2.15 | 1.55 |
| RCI score (3-month) | PND | 183 | -1.245 | 0.6705 | -3.808 | -0.503 |
| | No PND | 420 | 0.6022 | 0.7966 | -0.4966 | 3.67 |

NB. Negative values = worse performance.

A Pearson correlation coefficient was computed to assess the linear relationship between the combined episodic and working memory risk score (pre-surgery) and PND RCI outcome score at 3 months: r = 1.54, p<0.001 value. The direction of results indicates poorer performance at baseline on the combined episodic and working memory risk score to be significantly associated with a negative change from baseline RCI score at 3 months post-surgery. This direction of result indicates that worse performance on the combined episodic and working memory risk score is associated with a decline in cognitive performance post-surgery. To further explore this association, binary logistic regressions were performed to confirm poorer baseline performance (i.e., having a pre-surgical cognitive impairment relative to the normative mean) as a significant predictor of risk for developing PND.

Binary logistic regression indicates overall pre-surgical cognitive impairment on the combined episodic + working memory risk score (i.e., cognitive test performance below the mean of the normative comparison) is a significant predictor of PND (negative RCI score), when controlling for age: Wald=11.472, P<0.001. The odds ratio for the combined risk score is 1.764 (95% Cl 1.270 - 2.450), indicating that the odds of having PND at 3 months are =100(1.764 - 1) = 76% higher for those with worse than normative mean performance pre-surgery on the combined risk score, than those performing better than the normative mean pre-surgery.

Binary logistic regression indicates overall pre-surgical cognitive impairment on the combined episodic + working memory risk score (i.e., cognitive test performance below the mean of the normative comparison) is a trending significant predictor of PND (RCI score >0.5SD), when controlling for age: Wald=3.114, P=0.078. The odds ratio for the risk score is 1.379 (95% Cl 0.965 - 1.971), indicating that the odds of having PND at 3 months are =100(2.064 - 1) = 38% higher for those with worse than normative mean performance pre-surgery on the combined risk score, than those performing better than the normative mean pre-surgery. The percentage of participants meeting PND as defined by RCI score >0.5SD decline from baseline is 30%.

Out of the patients examined, 6% had a mild-to-moderate pre-surgical impairment as defined by performance >1SD below the normative comparison on the combined episodic + working memory risk score. Binary logistic regression indicates that pre-surgical deficit of >1SD below the mean of the normative comparison on the combined episodic + working memory risk score is trending as a significant predictor of PND (negative RCI score), when controlling for age: Wald=3.048, P=0.081. The odds ratio for the risk score is 1.877 (95% Cl 0.926 - 3.805). indicating that the odds of having PND at 3 months are =100(1.877 - 1) = 88% higher for those with a >1 SD pre-surgical deficit on the combined risk score than those with no pre-surgical deficit. Binary logistic regression indicates that pre-surgical deficit of >1SD below the mean of the normative comparison on the combined episodic + working memory risk score is a significant predictor of PND (RCI score >0.5SD), when controlling for age: Wald=4.231, P=0.040. The odds ratio for the risk score is 2.064 (95% Cl 1.035 - 4.118), indicating that the odds of having PND at 3 months are =100(2.064 - 1) = 106% higher for those with a >1SD pre-surgical deficit on the combined risk score than those with no pre-surgical deficit. The percentage of participants meeting PND as defined by RCI score >0.5SD decline from baseline is 30%.

A secondary analysis of a large European dataset successfully identified cognitive risk factors (episodic and working memory) for determining PND outcome, whilst controlling for age. The outcome of this analysis provides support for the development of a standardized prediction tool to measure key factors associated with risk for PND, providing major medical advancement to this field.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention. As such, the specific embodiments are not intended to be exhaustive or to limit the invention to the precise forms disclosed. Therefore, it should be understood that embodiments of the disclosed subject matter are in no way limited to a particular element, compound, composition, component, article, apparatus, methodology, use, protocol, step, and/or limitation described herein, unless expressly stated as such.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims.

Furthermore, those of ordinary skill in the art will recognize that certain changes, modifications, permutations, alterations, additions, subtractions and sub-combinations thereof can be made in accordance with the teachings herein without departing from the spirit of the present invention. Furthermore, it is intended that the following appended claims and claims hereafter introduced are interpreted to include all such changes, modifications, permutations, alterations, additions, subtractions and sub-combinations as are within their true spirit and scope. Accordingly, the scope of the present invention is not to be limited to that precisely as shown and described by this specification.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/-0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators - such as, e.g., “first,” “second,” “third,” etc. - for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

Lastly, all patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A method of treating a subject at risk for developing perioperative neurocognitive disorder, prior to surgery or other operation, comprising:
a) obtaining an episodic memory index, a working memory index, or a combined episodic memory and a working memory index for the subject;
wherein the episodic memory index is obtained by measuring the episodic memory of the subject by having the subject perform the episodic memory test Cambridge neuropsychological Test Automated Battery (CANTAB) Paired Associated learning (PAL) or CANTAB Verbal Recognition Memory (VRM) on a computational device
wherein the working memory index is obtained by measuring the working memory of the subject by having the subject perform the working memory test CANTAB Spatial Span (SSP) or Spatial Working Memory (SWM) on a computational device; and
wherein the combined episodic memory and working memory index is obtained by measuring both the episodic memory of the subject by having the subject perform the episodic memory test CANTAB PAL or CANTAB VRM and the working memory of the subject by having the subject perform the working memory test CANTAB-SSP or SWM on a computational device,
b) determining a surgical cognitive disorder risk-score by comparing the episodic memory index, the working memory index, or the combined episodic memory and working memory index to a normative mean;
c) identifying the subject as at medium risk for perioperative neurocognitive disorder or at high risk for perioperative neurocognitive disorder by comparing the surgical cognitive disorder risk-score to pre-defined threshold criteria
wherein the subject is at high risk for perioperative neurocognitive disorder when:
i) the episodic memory index is greater than or equal to a standard deviation of one below a normative mean; or
ii) the working memory index is greater than or equal to a standard deviation of one below a normative mean; or
iii) the combined episodic memory and working memory index is greater than or equal to a standard deviation of one below a normative mean, or
wherein the subject is at medium risk for perioperative neurocognitive disorder when:
i) the episodic memory index is below the normative mean but less than one standard deviation below the normative mean; or
ii) the working memory index is below the normative mean but less than one standard deviation below the normative mean; or
iii) the combined episodic memory and working memory index is below the normative mean but less than one standard deviation below the normative mean; and
d) treating the subject identified as being medium risk for perioperative neurocognitive disorder with one or more fibrates; and treating the subject identified as being high risk for perioperative neurocognitive disorder with one or more fibrates.

2. The method of claim 1 further comprising, prior to step (b), determining an index of one or more risk determinants for the perioperative neurocognitive disorder in the subject.

3. The method of claim 2, wherein determining a surgical cognitive disorder risk-score is done in conjunction with comparing the index for each of the one or more risk determinants to a normative mean.

4. The method of claim 2, wherein the one or more risk determinants comprise an inflammation index, an age index, a body mass index, an education index, a medicament use index, a disease diagnosis or history index, or any combination thereof.

5. The method of claim 1, wherein the surgical cognitive disorder risk-score is determined using predetermined threshold criteria of Table 1.

6. The method of claim 1, wherein the subject is identified as being at very high risk for a surgical cognitive disorder when:
a) the episodic memory index is greater than or equal to a standard deviation of one below a normative mean and the index of one or more risk determinants includes an inflammation index indicative of high inflammation, an age index of 60 or more years, a body mass index of 30 or more, an education index of 12 years of formal education or less, an medicament use index of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, a Parkinson’s medicament, or an anticholinergic-based medicament, and a disease diagnosis or history index of the presence of a psychiatric disorder, a neurological disorder, a diabetes, a metabolic syndrome, an autoimmune disease, a heart disease, a vascular disease, a stroke, an infection, an injury, an asthma, or alcohol abuse; or
b) the working memory index is greater than or equal to a standard deviation of one below a normative mean and the index of one or more risk determinants includes an inflammation index indicative of high inflammation, an age index of 60 or more years, a body mass index of 30 or more, an education index of 12 years of formal education or less, an medicament use index of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, a Parkinson’s medicament, or an anticholinergic-based medicament, and a disease diagnosis or history index of the presence of a psychiatric disorder, a neurological disorder, a diabetes, a metabolic syndrome, an autoimmune disease, a heart disease, a vascular disease, a stroke, an infection, an injury, an asthma, or alcohol abuse; or
c) the combined episodic memory and working memory index is greater than or equal to a standard deviation of one below a normative mean, and the index of one or more risk- related factors includes an inflammation index indicative of high inflammation, an age index of 60 or more years, a body mass index of 30 or more, an education index of 12 years of formal education or less, an medicament use index of use in the previous 8 weeks of an antihistamine, a gastrointestinal antispasmodic, an antivertigo/antiemetic, an antidepressant, a bladder antimuscarinic, a skeletal muscle relaxant, an antipsychotic, an antiarrhythmic, a Parkinson’s medicament, or an anticholinergic-based medicament, and a disease diagnosis or history index of the presence of a psychiatric disorder, a neurological disorder, a diabetes, a metabolic syndrome, an autoimmune disease, a heart disease, a vascular disease, a stroke, an infection, an injury, an asthma, or alcohol abuse; and
further comprising treating the subject identified as being very high risk for perioperative neurocognitive disorder with one or more fibrates.

7. The method of claim 1, wherein the subject is identified as being at high risk for a surgical cognitive disorder when:
a) the episodic memory index is greater than or equal to a standard deviation of one below a normative mean and an inflammation index of a blood level of 10 milligrams/liter or more of CRP; or
b) the working memory index is greater than or equal to a standard deviation of one below a normative mean and an inflammation index of a blood level of 10 milligrams/liter or more of CRP; or
c) the combined episodic memory and working memory index is greater than or equal to a standard deviation of one below a normative mean and an inflammation index of a blood level of 10 milligrams/liter or more of CRP; or
d) the episodic memory index is greater than or equal to a standard deviation of one below a normative mean and an age index of 60 or more years; or
e) the working memory index is greater than or equal to a standard deviation of one below a normative mean and an age index of 60 or more years; or
f) the combined episodic memory and working memory index is greater than or equal to a standard deviation of one below a normative mean and an age index of 60 or more years; and
wherein the method comprises treating the subject identified as being high risk for perioperative neurocognitive disorder with one or more fibrates.

8. The method of claim 1, wherein the subject is identified as being at medium risk for a surgical cognitive disorder when:
g) the episodic memory index is below the normative mean but less than one standard deviation below the normative mean and an inflammation index of a blood level of 10 milligrams/liter or more of CRP; or
h) the working memory index is below the normative mean but less than one standard deviation below the normative mean and an inflammation index of a blood level of 10 milligrams/liter or more of CRP; or
i) the combined episodic memory and working memory index that is below the normative mean but less than one standard deviation below the normative mean, and an inflammation index of a blood level of 10 milligrams/liter or more of CRP; or
j) the episodic memory index is below the normative mean but less than one standard deviation below the normative mean and an age index of 60 or more years; or
k) the working memory index is below the normative mean but less than one standard deviation below the normative mean and an age index of 60 or more years; or
I) the combined episodic memory and working memory index is below the normative mean but less than one standard deviation below the normative mean, and an age index of 60 or more years; and
wherein the method comprises treating the subject identified as being medium risk for perioperative neurocognitive disorder with one or more fibrates.

9. The method of claim 1, further comprising:
treating the subject identified as being medium risk for perioperative neurocognitive disorder with additional monitoring;
treating the subject identified as being medium risk for perioperative neurocognitive disorder before surgery;
treating the subject identified as being medium risk for perioperative neurocognitive disorder after surgery;
treating the subject identified as being high risk for perioperative neurocognitive disorder with additional monitoring;
treating the subject identified as being high risk for perioperative neurocognitive disorder before surgery; or
treating the subject identified as being high risk for perioperative neurocognitive disorder after surgery.

10. The method of claim 1, further comprising:
identifying the subject as having a preoperative neurocognitive disorder and treating the subject identified as having the preoperative neurocognitive disorder with one or more fibrates,
identifying the subject as having a postoperative delirium and treating the subject identified as having the postoperative delirium with one or more fibrates,
identifying the subject as having a delayed postoperative neurocognitive recovery and treating the subject identified as having the delayed postoperative neurocognitive recovery with one or more fibrates,
identifying the subject as having a mild postoperative neurocognitive disorder and treating the subject identified as having the mild postoperative neurocognitive disorder with one or more fibrates,
identifying the subject as having a major postoperative neurocognitive disorder and treating the subject identified as having the major postoperative neurocognitive disorder with one or more fibrates,
identifying the subject as having a mild neurocognitive disorder and treating the subject identified as having the mild neurocognitive disorder with one or more fibrates, or
identifying the subject as having a major neurocognitive disorder and treating the subject identified as having the major neurocognitive disorder with one or more fibrates.

11. The method of any one of claim 1, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

12. A method of treating a subject with post-viral cognitive impairment, comprising:
a) obtaining an episodic memory index, a working memory index, or a combined episodic memory and a working memory index for the subject;
wherein the episodic memory index is obtained by measuring the episodic memory of the subject by having the subject perform the episodic memory test Cambridge Neuropsychological Test Automated Battery (CANTAB) Paired Associated learning (PAL) or CANTAB Verbal Recognition Memory (VRM) on a computational device,
wherein the working memory index is obtained by measuring the working memory of the subject by having the subject perform the working memory test CANTAB Spatial Span (SSP) or Spatial Working memory (SWM) on a computational device; and
wherein the combined episodic memory and working memory index is obtained by measuring both the episodic memory of the subject by having the subject perform the episodic memory test CANTAB PAL, or CANTAB VRM and the working memory of the subject by having the subject perform the working memory test CANTAB-SSP or SWM;
b) obtaining a post-viral cognitive impairment diagnostic-index by comparing the episodic memory index, the working memory index, or the combined episodic memory and a working memory index to a normative mean; and
c) identifying the subject as having post-viral cognitive impairment by comparing the post-viral cognitive impairment diagnostic-score to pre-defined threshold criteria.
wherein the subject has post-viral cognitive impairment when:
i) the episodic memory index is greater than or equal to a standard deviation of one below a normative mean;
ii) the working memory index is greater than or equal to a standard deviation of one below a normative mean; or
iii) the combined episodic memory and working memory index is greater than or equal to a standard deviation of one below a normative mean; and
d) treating the subject with post-viral cognitive impairment with one or more fibrates.

13. The method of claim 12, further comprising, prior to step (b), determining a index of one or more risk determinants for the post-viral cognitive impairment in the subject.

14. The method of claim 13, wherein determining the post-viral cognitive impairment diagnostic-index is done in conjunction with comparing the index for each of the one or more risk determinants to a normative mean.

15. The method of claim 13, wherein the one or more risk determinants comprise an inflammation index, an anti-viral medicament use index, a viral infection diagnosis or history index, or any combination thereof.

16. The method of claim 12, wherein the wherein the post-viral cognitive impairment diagnostic-index is determined using predetermined threshold criteria of Table 2.

17. The method of claim 13, wherein the subject has post-viral cognitive impairment when:
a) the episodic memory index is greater than or equal to a standard deviation of one below a normative mean and there are one or more risk determinants present; or
b) the working memory index is greater than or equal to a standard deviation of one below a normative mean and there are one or more risk determinants s present; or
c) the combined episodic memory and working memory index is greater than or equal to a standard deviation of one below a normative mean and there are one or more risk determinants present; or
d) the episodic memory index is below the normative mean but less than one standard deviation below the normative mean and there are one or more risk determinants s present; or
e) the working memory index is below the normative mean but less than one standard deviation below the normative mean and there are one or more risk determinants present; or
f) the combined episodic memory and working memory index is below the normative mean but less than one standard deviation below the normative mean and there are one or more risk determinants present.

18. The method of claim 12, wherein the one or more fibrates include bezafibrate, ciprofibrate, clinofibrate, clofibrate, clofibride, fenofibrate, fenofibric acid, gemfibrozil, ronifibrate, simfibrate, or any combination thereof.

19. A composition comprising one or more fibrates for use in treating a subject at high risk or medium risk for a perioperative neurocognitive disorder,
wherein a subject at high risk or medium risk for a perioperative neurocognitive disorder is identified by a method comprising:
obtaining an episodic memory index, a working memory index, or a combined episodic memory and a working memory index for the subject;
wherein the episodic memory index is obtained by measuring the episodic memory of the subject by having the subject perform the episodic memory test Cambridge Neuropsychological Test Automated Battery (CANTAB) Paired Associated learning (PAL) or CANTAB Verbal Recognition Memory (VRM) on a computational device;
wherein the working memory index is obtained by measuring the working memory of the subject by having the subject perform the working memory test CANTAB Spatial Span (SSP) or Spatial Working Memory (SWM) on a computational device; and
wherein the combined episodic memory and working memory index is obtained by measuring both the episodic memory of the subject by having the subject perform the episodic memory test CANTAB PAL, or CANTAB VRM and the working memory of the subject by having the subject perform the working memory test CANTAB-SSP or SWM; and
b) determining a surgical cognitive disorder risk-score by comparing the episodic memory index, the working memory index, or the combined episodic memory and working memory index to a normative mean; and
c) identifying the subject as at medium risk for perioperative neurocognitive disorder or at high risk for perioperative neurocognitive disorder by comparing the surgical cognitive disorder risk-score to pre-defined threshold criteria:
wherein the subject is at high risk for a surgical cognitive disorder when:
i) the episodic memory index is greater than or equal to a standard deviation of one below a normative mean; or
ii) the working memory index is greater than or equal to a standard deviation of one below a normative mean; or
iii) the combined episodic memory and working memory index is greater than or equal to a standard deviation of one below a normative mean, or
wherein the subject is at medium risk for a surgical cognitive disorder when:
i) the episodic memory index is below the normative mean but less than one standard deviation below the normative mean; or
ii) the working memory index is below the normative mean but less than one standard deviation below the normative mean; or
iii) the combined episodic memory and working memory index is below the normative mean but less than one standard deviation below the normative mean.

20. A composition comprising one or more fibrates for use in treating a subject with a post-viral cognitive impairment, wherein a subject with post-viral cognitive impairment is identified by the method comprising:
a) obtaining an episodic memory index, a working memory index, or a combined episodic memory and a working memory index for the subject;
wherein the episodic memory index is obtained by measuring the episodic memory of the subject by having the subject perform the episodic memory test Cambridge Neuropsychological Test Automated Battery (CANTAB) Paired Associated learning (PAL) or CANTAB Verbal Recognition Memory (VRM) on a computational device,
wherein the working memory index is obtained by measuring the working memory of the subject by having the subject perform the working memory test CANTAB Spatial Span (SSP) or Spatial Working memory (SWM) on a computational device; and
wherein the combined episodic memory and working memory index is obtained by measuring both the episodic memory of the subject by having the subject perform the episodic memory test CANTAB PAL, or CANTAB VRM and the working memory of the subject by having the subject perform the working memory test CANTAB-SSP or SWM;
b) obtaining a post-viral cognitive impairment diagnostic-index by comparing the episodic memory index, the working memory index, or the combined episodic memory and a working memory index to a normative mean; and
c) identifying the subject as having post-viral cognitive impairment by comparing the post-viral cognitive impairment diagnostic-score to pre-defined threshold criteria;
wherein the subject has post-viral cognitive impairment when:
i) the episodic memory index is greater than or equal to a standard deviation of one below a normative mean;
ii) the working memory index is greater than or equal to a standard deviation of one below a normative mean; or
iii) the combined episodic memory and working memory index is greater than or equal to a standard deviation of one below a normative mean; and
d) treating the subject with post-viral cognitive impairment with one or more fibrates.
